SEQUENCE LISTING (1 sequence)

SEQ ID NO: 1 - DNA, 24 bp, Artificial sequence
  Synthetic primer
ctagaagcttctagaagcttctag

(19) United States
(12) Patent Application Publication
(10) Pub. No.: US 2007/0128303 A1
(43) Pub. Date: Jun. 7, 2007

(54) Anti-inflammatory, cytoprotective factor derivable from a probiotic organism

(75) Inventors: Eugene B. Chang, Chicago, IL (US); Elaine O. Petrof, Chicago, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE UNIVERSITY OF CHICAGO, Chicago, IL (US)

(21) Appl. No.: 10/589,544

(22) PCT Filed: Feb. 4, 2005
(86) PCT No.: PCT/US05/03765

§ 371 Date: Jan. 19, 2007

Related U.S. Application Data
(60) Provisional application No. 60/542,725, filed on Feb. 6, 2004.

(51) Int. Cl. A61K 35/74

(52) U.S. Cl. 424/780

(57) ABSTRACT

The invention provides an isolated, anti-inflammatory, cytoprotective compound that is soluble in aqueous fluid, is derivable from the conditioned medium of a probiotic culture, such as VSL#3, induces heat shock protein expression, and has shown the capacity to inhibit NF-κB activation. The compound is amenable to formulation in a pharmaceutical composition and to packaging in a kit form with instructions for use in methods according to the invention, which include methods of preventing, treating, or ameliorating a symptom of an inflammatory disorder, such as an inflammatory epithelial disease, e.g., inflammatory bowel disease, characterized by inflammation.

The government owns rights in the invention pursuant to grant numbers DK47722, DK42086, T32 GM07019, and K08 DK064840-01 from the National Institutes of Health.

### FIELD OF THE INVENTION

The invention relates generally to the field of inflammatory disorders. More particularly, it concerns inflammatory bowel diseases, such as ulcerative colitis and Crohn's disease.

### BACKGROUND OF THE INVENTION

Inflammatory bowel disease (IBD) is a group of chronic disorders, such as ulcerative colitis and Crohn's disease, that cause inflammation or ulceration of the digestive tract. The unfortunate combination of genetic background, exposure to environmental factors, or colonization by certain inciting commensal bacteria, can result in the development of IBD in susceptible individuals.

Ulcerative colitis causes inflammation and ulceration of the inner lining of the colon and rectum. It rarely affects the small intestine except for the end that connects to the colon, called the terminal ileum. Ulcerative colitis may also be called colitis or proctitis. Ulcerative colitis may occur in people of any age, but most often it starts between ages 15 and 30. Ulcerative colitis affects men and women equally and appears to run in some families. Theories about what causes ulcerative colitis abound, but none have been proven. A popular theory is that the body's immune system reacts to a virus or a bacterium by causing ongoing inflammation in the intestinal wall.

The most common symptoms of ulcerative colitis are abdominal pain and bloody diarrhea. Patients also may experience fatigue, weight loss, loss of appetite, rectal bleeding, and loss of body fluids and nutrients. About half of patients have mild symptoms. Others suffer frequent fever, bloody diarrhea, nausea, and severe abdominal cramps. Ulcerative colitis may also cause problems such as arthritis, inflammation of the eye, liver disease (hepatitis, cirrhosis, and primary sclerosing cholangitis), osteoporosis, skin rashes, and anemia. No one knows for sure why problems occur outside the colon. Scientists think these complications may occur when the immune system triggers inflammation in other parts of the body. Some of these problems go away when the colitis is treated.

The extent and severity of mucosal injury in inflammatory bowel diseases are determined by the disequilibrium between two opposing processes, reparative and cytoprotective mechanisms versus inflammation-induced injury.

Treatment for ulcerative colitis depends on the seriousness of the disease. Most people are treated with medication. In severe cases, a patient may need surgery to remove the diseased colon. Some people whose symptoms are triggered by certain foods are able to control the symptoms by avoiding foods that upset their intestines, like highly seasoned foods, raw fruits and vegetables, or milk sugar (lactose). Some people have remissions that last for months or even years. However, most patients' symptoms eventually return.

The goal of therapy is to induce and maintain remission, and to improve the quality of life for people with ulcerative colitis. Several types of drugs are currently available.

Aminosalicylate drugs, such as those that contain 5-aminosalicylic acid (5-ASA), help control inflammation. Sulfasalazine is a combination of sulfapyridine and 5-ASA and is used to induce and maintain remission. The sulfapyridine component carries the anti-inflammatory 5-ASA to the intestine. However, sulfapyridine may lead to side effects such as nausea, vomiting, heartburn, diarrhea, and headache. Other 5-ASA agents such as olsalazine, mesalamine, and balsalazide, have a different carrier, offer fewer side effects, and may be used by people who cannot take sulfasalazine. 5-ASAs are given orally, through an enema, or in a suppository, depending on the location of the inflammation in the colon. Most people with mild or moderate ulcerative colitis are treated with this group of drugs first.

Corticosteroids, such as prednisone and hydrocortisone, also reduce inflammation. They may be used by people who have moderate to severe ulcerative colitis or who do not respond to 5-ASA drugs. Corticosteroids can be given orally, intravenously, through an enema, or in a suppository. These drugs can cause side effects such as weight gain, acne, facial hair, hypertension, mood swings, and an increased risk of infection. For this reason, they are not recommended for long-term use.

Immunomodulators, such as azathioprine and 6-mercapto-purine (6-MP), reduce inflammation by affecting the immune system. They are used for patients who have not responded to 5-ASAs or corticosteroids or who are dependent on corticosteroids. However, immunomodulators are slow-acting and it may take up to 6 months before the full benefit is seen. Patients taking these drugs are monitored for complications including pancreatitis and hepatitis, a reduced white blood cell count, and an increased risk of infection. Cyclosporine A may be used with 6-MP or azathioprine to treat active, severe ulcerative colitis in people who do not respond to intravenous corticosteroids.

In addition to the above, other drugs may be given to relax the patient or to relieve pain, diarrhea, or infection.

About 25-40% of ulcerative colitis patients must eventually have their colons removed because of massive bleeding, severe illness, rupture of the colon, or risk of cancer. Sometimes the doctor will recommend removing the colon if medical treatment fails or if the side effects of corticosteroids or other drugs threaten the patient's health.

Crohn's disease differs from ulcerative colitis in that it may affect any part of the digestive tract. It causes inflammation and ulcers that may affect the deepest layers of lining of the digestive tract. Anti-inflammatory drugs, such as 5-aminosalicylates (e.g., mesalamine) or corticosteroids, are typically prescribed, but are not always effective. Immunosuppression with cyclosporine is sometimes beneficial for patients resistant to or intolerant of corticosteroids.

Nevertheless, surgical correction is eventually required in 90% of patients with Crohn's disease; 50% undergo colonic resection. (Leiper et al, 1998; Makowiec et al., 1998). The recurrence rate after surgery is high, with 50% requiring further surgery within 5 years. (Leiper et al., 1998; Besnard et al., 1998).

Current concepts regarding the etiopathogenesis of IBD suggest that there is a disequilibrium between the processes of cytoprotection and wound healing and the pro-inflammatory pathways, the net result of which culminates in a state of proinflammatory overactivity and resultant damage to the intestinal mucosa (Chang, 1999; Podolsky, 2002). Central to preserving mucosal integrity is maintenance of epithelial barrier function, as evidenced by the fact that altered tight junction structure resulting in impaired barrier function is thought to contribute to the clinical sequelae of ulcerative colitis (Schmitz et al., 1999).

Through the use of sense and antisense transfection experiments, it has been shown that heat shock proteins play a central role in providing cytoprotection to epithelial cells, as illustrated by their ability to protect epithelial barrier function under conditions of oxidative stress (Ropeleski et al, 2003; Urayama et al., 1998). Inducible heat shock proteins (Hsp) belong to a family of highly conserved proteins that play an important role in protecting cells against physiologic and pathogenic stressors in the environment. Under conditions of stress such as heat, exposure to heavy metals, and toxins, ischemia/reperfusion injury, or oxidative stress from inflammation, Hsp induction is both rapid and robust. Induction of heat shock proteins by a mild “stress” confers protection against subsequent insult or injury, which would otherwise lead to cell death. This well-described phenomenon is known as “stress tolerance” (Parsell and Lindquist, 1993).

In intestinal epithelial cells, inducible heat shock proteins convey a degree of cytoprotection against stressors such as inflammatory cell-derived oxidants and preserve the integrity of intestinal epithelial cell barrier function under hostile conditions (Chang, 1999; Musch et al., 1996; Musch et al., 1999). The induction of heat shock proteins in intestinal epithelial cells prolongs viability under conditions of stress (Musch et al., 1996) and preserves tight junctions as measured by transepithelial resistance (Musch et al., 1999).

Activation of the pro-inflammatory NF-κB pathway is thought to be a key molecular event involved in the pathogenesis of IBD (Neurath et al, 1998; Jobin and Sartor, 2000; Schmid and Adler, 2000; Boone et al., 2002). Administration of antisense oligonucleotides targeting the NF-κB subunit p65 was more effective than steroid treatment in reducing inflammation in two different murine models of colitis (Neurath et al., 1996). Immunohistochemical studies have shown that colonic biopsies from Crohn's patients display increased levels of expression of the NF-κB subunit p65 in areas of active inflammation (Neurath et al., 1998). In the non-inflammatory state, NF-κB is held in its inactive, cytosolic form complexed to the inhibitory protein IκB. Once a signal is received to activate NF-κB, its inhibitor IκB is phosphorylated and targeted for degradation by the ubiquitin proteasome pathway. The release of NF-κB from inhibition and its translocation to the nucleus, results in the transcriptional activation of a broad spectrum of cytokine and chemokine genes, cell adhesion molecules, and immunoreceptors, all important mediators of the inflammatory response (Neurath et al, 1998; Jobin and Sartor, 2000; Schmid and Adler, 2000; Boone et al., 2002).

There is growing interest in the use of probiotics, which are defined as ingestible microorganisms having health benefit beyond their intrinsic nutritive value, in the treatment of a variety of gastrointestinal ailments including inflammatory bowel diseases (Gionchetti et al., 2000a), irritable bowel syndrome (Niedzielin et al., 2001), pouchitis (Gionchetti et al., 2000b; Gionchetti et al., 2003), as well as rotavirus and antibiotic-associated diarrhea (Isolauri et al., 1991; Majamaa et al., 1995; Arvola et al., 1999). Although little is known about their mechanisms of action, probiotics appear to have protective, trophic, and anti-inflammatory effects on bowel mucosa.

Proposed mechanisms by which probiotics may act include the production of ammonia, hydrogen peroxide (Kullisaar et al., 2002; Annuk et al., 2003; Ocana et al, 1999), and bacteriocins (Cleveland et al., 2001; Paraje et al., 2000; Braude and Siemienski, 1968), which inhibit the growth of pathogenic bacteria, the competition for adhesion sites on intestinal epithelia (Lee et al., 2000; Lee et al., 2003), and an adjuvant-like stimulation of the immune system against pathogenic organisms (Maassen et al., 2000). However, the exact mechanisms by which probiotics act to protect against intestinal inflammation have yet to be fully elucidated.

The probiotic VSL#3 (comprised of Streptococcus thermophilus, and several species of Lactobacillus and Bifidobacteria) attenuates intestinal inflammation in the IL-10 knockout mouse model of enterocolitis (Madsen et al, 2001) and has been shown to improve the clinical outcome of chronic intestinal inflammation in clinical trials (Gionchetti et al., 2000b). In a randomized, double-blinded, placebo-controlled trial of 40 patients suffering from at least 3 relapses per year of recurrent pouchitis, those patients assigned to receive placebo all relapsed within four months, whereas only 15% ( 3/20) of the patients assigned to the probiotic treatment arm developed relapse (Gionchetti et al., 2000b). In addition to maintenance therapy, VSL#3 as prophylactic treatment may help prevent the onset of acute pouchitis in the year following ileal pouch-anal anastomosis after colectomy for ulcerative colitis (Gionchetti et al., 2003).

Changing the gut flora of IBD patients with probiotic agents is being intensely studied as a therapeutic strategy. However, the mechanisms of probiotic action remain unclear. Moreover, the clinical efficacy of probiotics is highly dependent on the ability to establish and maintain bacterial colonization, and is limited by unregulated composition of formulations and homeopathic delivery of active agents. Thus, there is a need to elucidate the mechanisms of probiotic activity and develop more effective therapies for inflammatory bowel diseases.

### SUMMARY OF THE INVENTION

The invention disclosed herein satisfies at least one of the aforementioned needs in the art by providing at least one soluble factor from the probiotic VSL#3, wherein the soluble factor(s) is useful in treating or preventing inflammatory disorders, such as inflammatory bowel disease. The soluble factor(s) inhibits the chymotrypsin-like activity of the proteasome in, e.g., intestinal epithelial cells. Proteasome inhibition occurs relatively soon after exposure of the epithelial cells to the probiotic-conditioned medium containing the soluble factor(s). In addition, the conditioned medium has shown a capacity to inhibit the pro-inflammatory NF-κB pathway, and does it through a mechanism different from type-III secretory mechanisms that have been described. The soluble factor(s) also induces expression of cytoprotective heat shock proteins (Hsp, e.g., Hsp25 and Hsp72) in intestinal epithelial cells. Without wishing to be bound by theory, these effects appear to be mediated through the common unifying mechanism of proteasome inhibition. The resulting inhibition of NF-κB and increased expression of one or both of Hsp25 and Hsp72 are consistent with the anti-inflammatory and cytoprotective effects of the soluble factor(s) and reveal a new mechanism underlying microbial-epithelial interaction.

The invention provides bioactive compounds or agents secreted by probiotic bacteria that attenuate the TNF-α-mediated induction of NF-κB activation in intestinal epithelial cells and induce the expression of cytoprotective heat shock proteins, thus affecting at least one, and perhaps two, “arms” of current inflammatory bowel disease models. These beneficial effects on the gut mucosa appear to stem from a common mechanism mediated by proteasome inhibition. The compounds of the invention provide the basis for therapies for the treatment of IBD that are superior to those currently available in the art.

In one aspect of the invention, a composition is provided that comprises an isolated, anti-inflammatory, cytoprotective compound. In an embodiment, the compound is present in a probiotic-conditioned medium. One suitable probiotic-conditioned medium is medium conditioned by the probiotic, VSL#3. Preferably, the compound is present in an ether-extracted fraction of the probiotic-conditioned medium.

In some embodiments, the compound is an organic acid. In other embodiments, the invention provides an isolated, anti-inflammatory, cytoprotective compound comprised in medium conditioned with one or more of Streptococcus salivarius subsp. thermophilus, Lactobacillus casei, Lactobacillus plantarum, Lactobacillus acidophilus, Lactobacillus delbrueckii subsp. bulgaricus, Bifidobacteria longum, Bifidobacteria infantis, and Bifidobacteria breve. In some embodiments, the compound is present in a medium conditioned with Lactobacillus plantarum and in other embodiments, the conditioned medium is VSL#3-conditioned medium.

In another aspect of the invention, the compound induces the expression of at least one heat shock protein. In a particular embodiment, the heat shock protein is at least one of Hsp25 and Hsp72. In another embodiment the compound is an inhibitor of NF-κB activation, such as by inhibiting the NF-κB pathway. Preferably, the compound inhibits the NF-κB pathway by stabilizing IκB, such as by stabilizing unphosphorylated IκB, phosphorylated IκB, or both forms of IκB. In yet other embodiments, the compound is both an inducer of heat shock protein expression and an inhibitor of the NF-κB pathway.

In another aspect of the invention, the compound is a proteasome inhibitor. The compound may be a selective inhibitor of the proteasome. The selectivity of the proteasome inhibitor may be with regard to the protease activity of the proteasome, the type of cells in which it inhibits the proteasome, or both. In one embodiment of this aspect of the invention, the compound selectively inhibits the chymotrypsin-like activity of the proteasome. In other embodiments, the compound does not significantly inhibit the trypsin-like activity of the proteasome. In yet other aspects of the invention the compound weakly inhibits the caspase-like activity of the proteasome, wherein “weak inhibition” refers to a level of inhibition equivalent to that caused by 10 μM lactacystin. In still other embodiments, the compound selectively inhibits the proteasome in epithelial cells. Preferably, the compound selectively inhibits the proteasome in intestinal, or gut, epithelial cells.

Another aspect of the invention is drawn to a pharmaceutical composition comprising an isolated, anti-inflammatory, cytoprotective compound derived from a probiotic-conditioned medium and at least one pharmaceutically acceptable excipient. An exemplary pharmaceutical composition comprises an isolated, anti-inflammatory, cytoprotective compound derived from an ether-extracted fraction of a conditioned medium, such as a VSL#3-conditioned medium. In some embodiments, the compound is an organic acid. In some embodiments, the compound induces expression of at least one heat shock protein, e.g., Hsp25 and/or Hsp72. In an illustrative embodiment, the compound is an inhibitor of NF-κB activation, such as by stabilizing IκB, whether phosphorylated IκB or not. In other embodiments, the compound is a proteasome inhibitor, such as a selective inhibitor of the chymotrypsin-like activity of a proteasome. An exemplary proteasome is an epithelial cell proteasome, such as an intestinal epithelial cell proteasome.

Yet another aspect of the invention is a method for treating a patient with an inflammatory disorder comprising administering to the patient an effective amount of an isolated, anti-inflammatory, cytoprotective compound derived from a probiotic-conditioned medium. Typically, the compound is administered in an amount effective to slow, halt or reverse the progress of an inflammatory disorder, such as an inflammatory disease or condition; however, also contemplated is the administration of a compound as described herein in an amount effective to ameliorate a symptom associated with an inflammatory disorder. Symptoms associated with inflammatory disorders, such as redness, swelling, heat and pain, are known in the art, as are methods for measuring or assessing such a symptom to determine whether that symptom has been ameliorated. The inflammatory disorder may be an autoimmune disorder. Examples of autoimmune disorders that may be treated according to the invention include rheumatoid arthritis, juvenile rheumatoid arthritis, osteoarthritis, psoriatic arthritis, atopic dermatitis, eczematous dermatitis, psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, leprosy reversal reactions, erythema nodosum leprosum, autoimmune uveitis, polychondritis, Stevens-Johnson syndrome, lichen planus, sarcoidosis, primary biliary cirrhosis, uveitis posterior, or interstitial lung fibrosis.

In a preferred embodiment of this aspect of the invention, the inflammatory disorder is an inflammatory bowel disease. In one aspect of the invention the inflammatory bowel disease is Crohn's disease. In some embodiments, the inflammatory bowel disease is ulcerative colitis. A preferred probiotic-conditioned medium for use in this aspect of the invention is a VSL#3-conditioned medium. In some embodiments of this aspect of the invention, the compound is derived from an ether-extracted fraction of the medium (i.e., the compound is extracted from the medium using ether). It is contemplated that compounds useful in the practice of the method will include organic acids and acid-stable proteins or peptides. In some embodiments, the compound induces the expression of at least one heat shock protein, such as Hsp25 and/or Hsp72. The compound may inhibit NF-κB activation (e.g., by stabilizing IκB in a phosphorylated or unphosphorylated form) without or, preferably, with the induction of at least one heat shock protein. In some embodiments, the compound used in the method is an inhibitor of a protease activity, such as a protease activity of a proteasome. For example, a compound used in the method may selectively inhibit the chymotrypsin-like activity of a proteasome, such as an epithelial cell proteasome (e.g., an intestinal epithelial cell proteasome). Embodiments according to this aspect of the invention include the method of treating a patient with an inflammatory disorder wherein the anti-inflammatory, cytoprotective compound does not alter the ubiquitination level of at least one protein amenable to ubiquitination in an epithelial cell exposed to the compound.

A related aspect of the invention is directed to a method of preventing an inflammatory disorder comprising administering an effective amount of an anti-inflammatory, cytoprotective compound derived from a probiotic-conditioned medium. This aspect of the invention includes embodiments analogous to the above-described embodiments of treatment methods, with apparent modification of those embodiments to suit the prophylactic use of a compound according to the invention to prevent, rather than to treat, a patient with an inflammatory disorder.

Yet another aspect of the invention is drawn to a kit for treating (including ameliorating a symptom thereof) or preventing an inflammatory disorder comprising a pharmaceutical composition as described above and instructions for administration of the composition to treat or prevent the disorder.

Another aspect of the invention provides a method of producing an isolated, anti-inflammatory cytoprotective compound comprising obtaining a VSL#3-conditioned medium; and isolating an anti-inflammatory, cytoprotective compound from the VSL#3-conditioned medium, thereby producing an isolated, anti-inflammatory, cytoprotective compound. In some embodiments, the method further comprises characterizing the anti-inflammatory, cytoprotective compound. More preferably, the method further comprises identifying the anti-inflammatory, cytoprotective compound. In some embodiments, the method further comprises obtaining more anti-inflammatory, cytoprotective compound. In certain embodiments the more anti-inflammatory, cytoprotective compound is obtained by isolation from VSL#3. In other embodiments the more anti-inflammatory, cytoprotective compound is obtained by chemical synthesis. In yet other embodiments the method further comprises placing the more anti-inflammatory, cytoprotective compound in a pharmaceutical composition. In a preferred embodiment the method further comprises administering the pharmaceutical composition to a subject. Preferably the subject is a human. Also preferably, the subject has an inflammatory disorder. More preferably, the inflammatory disorder is an inflammatory bowel disease. In some embodiments the inflammatory bowel disease is Crohn's disease. In other embodiments the inflammatory bowel disease is ulcerative colitis.

Another aspect of the invention is drawn to a method of screening for a modulator of monocyte chemoattractant protein-1 (MCP-1) release, comprising: (a) combining a candidate modulator, a probiotic-conditioned medium, and an epithelial cell; (b) measuring MCP-1 release by the cell; and (c) comparing the MCP-1 release in the presence, and absence, of the candidate modulator, wherein a difference in the MCP-1 release identifies the candidate modulator as a modulator of MCP-1 release.

In a related aspect, the invention provides a method of screening for a modulator of heat shock protein expression, comprising (a) combining a candidate modulator, a probiotic-conditioned medium, and an epithelial cell; (b) measuring heat shock protein expression in said cell; and (c) comparing the heat shock protein expression in the presence, and absence, of said candidate modulator, wherein a difference in said heat shock protein expression identifies the candidate modulator as a modulator of heat shock protein expression. In some embodiments, the method will identify a modulator of expression of Hsp25 and/or Hsp72. Also contemplated are screening methods wherein the modulator alters the activity of Heat Shock Transcription Factor-1 (HSF-1).

Numerous additional aspects and advantages of the invention will become apparent to those skilled in the art upon consideration of the following detailed description of the invention, which describes presently preferred embodiments thereof.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIG. 1: Probiotic-conditioned medium inhibits TNF-alpha stimulation of NF-κB. YAMC (young adult mouse colon) cells were transfected with a NF-κB luciferase reporter gene and treated with VSL#3-conditioned medium (VSL#3-CM) for 16 hours, then stimulated with TNF-α (50 ng/ml 6 hours prior to harvest). Experimental conditions are as indicated below each column, “ctrl” column is untreated control, i.e., baseline level of NF-κB activity in YAMC cells prior to TNF-α stimulation. Transfections were performed in triplicate for each experimental condition. Shown is a representative graph from one of these experiments (n=8). Activity is expressed in arbitrary luminescence units. Results are normalized to the TK-Renilla reporter gene internal control, which is co-transfected with the NF-κB luciferase reporter gene in each experiment.

FIG. 2: Probiotic-conditioned medium stabilizes and prevents degradation of IκBα. Immunoblot of IκBα and the phosphorylated form of IκBα, 20 μg protein/lane. YAMC cells were treated with VSL#3-conditioned medium for 16 hours, then stimulated with TNF-α (50 ng/ml) and harvested at the times indicated. Shown in the upper two panels, TNF-α stimulates a transient phosphorylation of IκBα (5 minutes), is associated with decreased total IκBα (5-30 minutes) as IκBα is targeted for degradation. In the bottom panels, pretreatment of YAMC cells with VSL#3-conditioned medium inhibits the effects of TNF-α on IκBα and phosphorylated IκBα, preventing their degradation. Note the persistence of the phosphorylated form of IκBα (bottom panel).

FIG. 3: Global Ubiquitination is not inhibited by VSL#3-conditioned medium. Immunoblot analysis of ubiquitinated proteins from YAMC cells following treatment with VSL#3-CM for 16 hours, demonstrating that global blockade of ubiquitination does not occur when cells are treated with VSL#3-CM. MG132, a compound known to inhibit proteasome function and increase accumulation of ubiquitinated proteins, is also shown, as is thermal stress (HS) and untreated control cells (C). The pattern of ubiquitinated proteins observed after VSL#3-CM treatment most closely resembles the pattern seen with thermal stress. Molecular weight markers (kDa) are indicated to the right.

FIG. 4: Probiotic-conditioned medium inhibits proteasome activity. YAMC cells were treated with VSL#3-conditioned medium for 16 hours and then harvested for proteasome assay using the fluorogenic substrate SLLVY-AMC, which measures the chymotrypsin-like activity of the proteasome. Fluorescence is expressed in arbitrary units over time. Untreated control cells (-□-), cells treated with DH5α-CM (-∘-), VSL#3-CM (-▪-), and MG132 (-▴-) are indicated. As a positive inhibitor control MG132 was used at a concentration of 25 μM. Experimental conditions are as indicated, with data expressed as means and error bars expressed as standard errors of the mean (n=6).

FIG. 5: Hsp25 and Hsp72 expression is induced by probiotics, does not involve cell wall components, and is specific to epithelial cell types. FIG. 5A shows immunoblot analysis of levels of Hsp25 and Hsp72 in YAMC cells following exposure to VSL#3 bacteria for the times indicated, demonstrating a time-dependent increase in inducible Hsp expression. Last two lanes: 48 h=untreated controls harvested at 48 h, HS=heat shocked cells (positive control). Hsc73 (heat shock cognate 73), serves as a loading control.

FIG. 5B shows immunoblot analysis of levels of Hsp25 and Hsp72 in YAMC cells following exposure to VSL#3-conditioned medium or sonicated organisms at the concentrations of bacteria indicated (cfu/ml). Bacteria cultures were separated into either conditioned medium fraction (CM) or sonicated pellet (Pellet). A concentration-dependent increase in Hsp expression can be seen upon exposure to VSL#3-conditioned medium, which is not seen with sonicated pellet, indicating that the active factors produced by the bacteria are secreted into conditioned medium and are not cell wall components. Untreated cells are indicated (−), far left lane, and HS=heat shocked cells (positive control) are shown on far right lane. Hsc 73 serves as a loading control.

FIG. 5C shows immunoblot analysis of Hsp72, comparing different cell lines following exposure to VSL#3-conditioned medium for 16 hours, 20 μg protein/lane. VSL#3-conditioned medium induces a robust Hsp72 response in both colonic (YAMC) and small intestinal (MSIE) epithelial cells which is not seen in 3T3 fibroblast cells, suggesting that the probiotic effect is specific to epithelial cells. Untreated cells are indicated (−), thermal stress (HS) serves as a positive control.

FIG. 6: Probiotic compounds induce intestinal epithelial heat shock proteins through an apical (luminal) membrane specific process. YAMC intestinal epithelial cells exposed to VSL#3-conditioned medium from the apical (luminal) side demonstrate robust Hsp25 and Hsp72 protein expression. In contrast, cells exposed to VSL#3-conditioned medium from the basolateral side are not stimulated to express Hsp25 and Hsp72 proteins. When added to both sides, VSL#3-CM has a similar effect to what is seen when it is added only to the apical side. The constitutive heat shock cognate Hsc73 was used as a control.

FIG. 7: Time course of Hsp induction by the proteasome inhibitor MG132 is similar to that produced by VSL#3-conditioned medium. Immunoblot analysis of Hsp25 and Hsp72 levels in YAMC cells (20 μg protein/lane) following exposure to the proteasome inhibitor MG132 (25 μM) for the times indicated, demonstrating a time-dependent increase in Hsp expression which parallels that seen with VSL#3-treated cells. First two lanes: C=untreated control cells harvested at 0 h, V=DMSO vehicle-treated control harvested at 14 hours. HS=heat shocked cells (positive control). MG132 is even more effective at inducing Hsp25 than heat shock.

FIG. 8: Unlike MG132, treatment of epithelial cells with VSL#3-conditioned medium does not cause major toxicity. Phase-contrast photographs of YAMC cells treated with either VSL#3-CM (bottom left panel), DH5α-CM (bottom right panel), or MG132 at 25 μM (top right panel) for 16 hours, and untreated control cells (top left panel). Note the dramatic change in morphology and loss of cell viability in the MG132-treated cells. These changes are not seen in the VSL#3-CM or DH5α-CM treated cells, which look similar in appearance to untreated controls. Bar shown in top left panel equals 10 microns.

FIG. 9: The majority of bioactivity for the VSL#3-CM appears to reside in fractions that are less than 10 kDa. Hsp25 and Hsp72 protein expression is stimulated by components of VSL#3-CM that reside in fractions that were prepared through Centricon filters with a molecular weight cut-off of 10 kDa. The constitutive heat shock cognate Hsc73 was used as a control. Control (C), VSL#3-CM (CM), VSL#3-CM passed through 10 kDa filter (<110 kDa), heat shock (HS).

FIG. 10: VSL#3 bioactivity is pH-dependent. The induction of Hsp25 and Hsp72 by VSL#3-CM (CM) is influenced by the pH of the medium prior to its addition to the luminal fluid of YAMC monolayers. The pH values shown in the figure indicate the pH of the CM prior to its addition to the YAMC cells. Typically, the pH of the medium is 4.0 after being conditioned by the bacteria. The final pH after the 1:10 dilution in the luminal buffer is between 6.5 and 7.0, which is the approximate pH of the acid microclimate of intestinal epithelial cells in situ.

FIG. 11: Ether-extracted compounds of VSL#3-CM inhibit TNF-α-stimulated NF-κB activity. The effects of ether-extracted compounds (EEC) and MG132 on NF-κB activity were determined using an NF-κB ELISA assay (Active Motif). TNF-α stimulation (30 ng/ml) alone caused a significant increase in NF-κB activation (second bar from left). Both MG132 and EEC significantly inhibited TNF-stimulated NF-κB activity (third and fourth bar from left). In contrast, the remaining aqueous phase following separation from the ether fraction was devoid of activity (far right bar).

FIG. 12: Ether-extracted compounds of VSL#3-CM directly inhibit proteasomal function. The in vitro activity of the 20S proteasomal component (barrel) provided by the commercial proteasomal assay (Calbiochem) in the presence and absence of EEC from VSL#3 and E. coli (DH5α) was tested to determine if EEC directly inhibit proteasomal function. Proteasomal function was unaffected by EEC from DH5α (compare slopes). In contrast, there was significant inhibition of in vitro proteasomal activity by EEC from VSL#3 and MG132.

FIG. 13: Probiotic-conditioned medium displays differential inhibition of proteasome activity. YAMC cells were treated with VSL#3-conditioned medium for 16 hours and then harvested for proteasome assay using the fluorogenic substrate Bz-val-gly-arg-AMC (FIG. 13A) or Z-leu-leu-glu-AMC (FIG. 13B). Fluorescence is expressed in arbitrary units over time. Untreated control cells (-□-), cells treated with VSL#3-CM (-♦-), and lactacystin (- [symbol] -) are indicated. The data is expressed as means and error bars expressed as standard errors of the mean (n=3).

FIG. 14: FIG. 1. Probiotic-conditioned media inhibits TNF-alpha stimulation of NF-κB. YAMC cells were transfected with a NF-κB luciferase reporter gene and treated with VSL#3-conditioned media for 16 hours, then stimulated with TNF-α (50 ng/ml 6 hours prior to harvest). Experimental conditions are as indicated below each column. Transfections were performed in triplicate for each experimental condition (n=8), with the exception of the column showing VSL treatment alone, where data is compiled from three separate experiments, also performed in triplicate for each experiment. Data is expressed as mean±SE (*p<0.05 compared to TNF-α-treated samples). Activity is expressed in arbitrary luminescence units, normalized to the TK-Renilla internal control.

FIG. 15: Probiotic-conditioned media inhibits MCP-1 release. YAMC cells were treated with VSL#3-conditioned media (VSL-CM) for 16 hours, then stimulated with TNF-α (50 ng/ml) 6 hours prior to harvest and compared to untreated control (No Tx), TNF-α treatment alone (TNF-α only), or cells pretreated with conditioned media from the E. coli strain DH5α with and without TNF-α. Supernatants were assayed for release of the chemokine MCP-1 by ELISA (as described herein). Experimental conditions are as indicated below each column. YAMC cells pretreated with VSL-CM show a reduction in the amount of MCP-1 released in response to TNF-α stimulation compared to controls (mean±SE for three separate experiments, in each experiment each group was performed in triplicate, *p<0.05 compared to controls).

FIG. 16: Probiotic-conditioned media stabilizes and prevents degradation of IκBα. YAMC cells were treated with VSL#3-conditioned media for 16 hours, then stimulated with TNF-α (50 ng/ml) and harvested at the times indicated. Shown in the upper panel, TNF-α stimulates a transient phosphorylation of IκBα (5 minutes) and is associated with decreased total IκBα (5-15 minutes) as IκBα is targeted for degradation. In the bottom panel, pretreatment of YAMC cells with VSL#3-conditioned media inhibits the ability of TNF-α to stimulate IκBα degradation. Note the persistence of the phosphorylated form of IκBα.

FIG. 17: Probiotic-conditioned media modulates proteasome activity. VSL has a dramatic inhibitory effect on the chymotrypsin-like activity, no inhibitory effect on the trypsin-like activity, and a partial inhibitory effect on the caspase-like activity of the proteasome. Panel A: YAMC cells were treated with VSL#3-conditioned media for 16 hours and then analyzed for chymotrypsin-like proteasome activity. Fluorescence is expressed in arbitrary units over time. As a positive inhibitor control MG132 was used at a concentration of 25 μM, as described herein. Experimental conditions are as indicated, with data expressed as means and error bars expressed as standard errors of the mean (n=6). For the trypsin-like (Panel B) and caspase-like activities (Panel C), YAMC cells were treated with VSL#3-conditioned media for 16 hours and then analyzed as described but instead of MG132, the proteasome inhibitor lactacystin was used at a concentration of 10 μM (use of higher concentrations of lactacystin was limited due to cell toxicity). Data is expressed as means for three separate experiments, with error bars expressed as standard errors of the mean.

FIG. 18: Proteasome inhibition by probiotic-conditioned media is an early event. Time course of VSL#3-CM treatment demonstrating that proteasome inhibition by VSL#3-CM is an early event, occurring almost immediately after exposure of the epithelial cells to the probiotic-conditioned media. YAMC cells were treated for 30 minutes, 60 minutes, and 6 hours, then harvested and assayed for their ability to inhibit the CTL-like activity of the proteasome. Slopes of each assay, which represent degree of proteasome activity, were determined for each time point and plotted over time. The most pronounced proteasome inhibition occurs early after treatment with VSL#3-CM, with most of the inhibition occurring within the first 30 minutes. Shown is a graph representative of three separate experiments.

FIG. 19: Hsp25 and Hsp72 expression is induced by probiotics, does not involve cell wall components, and is specific to epithelial cell types. Panel A: Immunoblot analysis of levels of Hsp25 and Hsp72 in YAMC cells following exposure to VSL#3 bacteria for the times indicated, demonstrating a time-dependent increase in Hsp expression. Last two lanes: 48 h=untreated controls harvested at 48 hours, HS=heat-shocked cells (positive control). Hsc73 (heat shock cognate 73), serves as a loading control. Panel B: Immunoblot analysis of levels of Hsp25 and Hsp72 in YAMC cells following exposure to VSL#3-conditioned media or sonicated organisms at the concentrations of bacteria indicated (cfu/ml). Bacteria were grown as described herein, then separated into either a conditioned media fraction (CM) or a sonicated pellet fraction (Pellet). A concentration-dependent increase in Hsp expression can be seen upon exposure to VSL#3-conditioned media which is not seen with the sonicated pellet, indicating that the active factors or agents produced by the bacteria are secreted into conditioned medium and are not cell wall components. Hsc 73 serves as a loading control. Panel C: Immunoblot analysis of Hsp72, comparing different cell lines following exposure to VSL#3-conditioned media (CM) for 16 hours, 20 μg protein/lane. VSL#3-conditioned media induces a robust Hsp72 response in both colonic (YAMC) and small intestinal (MSIE) epithelial cells which is not seen in 3T3 fibroblast cells, suggesting that the probiotic effect is specific to epithelial cells. Thermal stress (HS) serves as a positive control.

FIG. 20: Hsp induction by probiotics is at least partly transcriptional and involves HSF-1. Panel A: Electrophoretic mobility shift assays (EMSA) show that the induction of Hsp expression by VSL#3-CM was transcriptional in nature. YAMC cells were treated for the times indicated with VSL#3-CM and then harvested, EMSAs were performed as described herein. VSL#3-CM induces binding of the heat shock transcription factor HSF, reaching a maximal signal around 4 or 5 hours after exposure and then tapering off after 6 hours, indicating that Hsp induction by VSL#3-CM is at least partly transcriptional in nature. Panel B: EMSA showing specificity of this binding by using antibodies against the transcription factors HSF-1 and HSF-2 (panel B). The major transcription factor involved in Hsp induction by VSL#3-CM is HSF-1; HSF-2 does not appear to play a role in this Hsp induction.

FIG. 21: Probiotic-conditioned media protects epithelial cells against oxidant stress. Panel A: Chromium release assay demonstrating that VSL#3-CM protects YAMC cells from oxidant injury. YAMC cells were treated with VSL#3-CM for 16 hours. Cells were labeled with ⁵¹Cr for 60 minutes and stimulated with monochloramine (NH₂Cl, 0.6 mM) for 60 minutes and the ratio of released ⁵¹Cr to intracellular ⁵¹Cr was determined (mean±SE for three separate experiments, in each experiment each group was performed in triplicate, *p<0.05 compared to controls). Panel B: VSL#3-CM prevents oxidant-induced actin depolymerization from the F-actin to the G-actin form. YAMC cells were treated with VSL#3-CM for 16 hours, when appropriate, and then treated with the oxidant monochloramine (0.6 mM, 60 minutes) along with untreated control (Con) cells. Cells were processed for globular (G) and filamentous (F) actin as described herein. Images shown are representative of three separate experiments.

### DETAILED DESCRIPTION

Inflammatory bowel diseases (IBDs) are a group of chronic disorders that affect the digestive tract of susceptible individuals. The extent and severity of mucosal injury in IBD is determined by the disequilibrium between inflammation-induced injury versus reparative and cytoprotective mechanisms. In recent in vitro and in vivo studies, various probiotics have been shown to be effective in either preventing or mitigating intestinal mucosal inflammation associated with experimental colitis (Madsen et al., 2001; Gionchetti et al., 2000b; Campierei et al., 2000). Furthermore, probiotics appear to reduce the rate of malignant transformation of colonic mucosa in the setting of chronic inflammation (Wollowski et al., 2001). A number of preliminary clinical trials have shown that probiotics are effective in the treatment of pouchitis and IBD. Several multicenter clinical trials are also under way to determine the effectiveness of these agents and to optimize dosage in IBD patients. The mechanism(s) of probiotic action, however, remains unclear. It follows that there is no appreciation in the art that the beneficial effects of crude probiotic materials, such as unrefined probiotic-conditioned media, can be ascribed to, and hence achieved, with one or more discrete compounds. Moreover, the therapeutic use of crude conditioned media of uncharacterized content presents significant health concerns.

The probiotic VSL#3 is disclosed herein as producing soluble factor(s) with anti-inflammatory and cytoprotective properties. More specifically, these factors inhibit the pro-inflammatory NF-κB pathway and induce the expression of cytoprotective heat shock proteins in intestinal epithelial cells. Moreover, these effects appear to be mediated through the common unifying mechanism of proteasome inhibition, although the invention is not contemplated as being limited by such explanatory theorizing. To facilitate a more thorough understanding of the invention, the following term definitions are provided.

“Isolated” in the context of describing the invention disclosed herein means that a given substance is separated from at least one other substance with which it is typically found in nature. By way of example, a bioactive agent “isolated” from a conditioned medium is separated from at least one other component of the relevant crude conditioned medium.

“Selective inhibition,” in the context of the selective inhibition of protease functions of the proteasome, means that less than all, and preferably one, protease function of a proteasome is reduced to a level comparable to the level of that protease measured in the presence of up to 10 μM lactocystin. For example, reduction of a chymotrypsin-like activity of a proteasome to a level found in the presence of no more than 10 μM lactocystin, without the concomitant reduction in the activity of at least one of the trypsin-like or the caspase-like proteasome activities, is illustrative of selective inhibition.

“Anti-inflammatory” has a plain meaning well known in the art as a substance or process that reduces inflammation, a physiological process generally characterized by heat, redness, swelling and pain. “Anti-inflammatory” is given its plain meaning herein.

“Inflammatory disorder” means any disease, malady, or condition known in the art to be characterized by involvement of inflammation. The term includes diseases, maladies and conditions of epithelial cells and, by way of particular example, of intestinal (i.e., gut) epithelial cells.

“Cytoprotective” has a plain meaning well known in the art as a substance or process that protects at least one cell or cell type, and it is this plain meaning that is given the term throughout this application.

“Probiotic-conditioned media” means a cell culture medium that has been exposed to viable cells. Suitable culture media include all media known in the art to be suitable for the growth, and/or maintenance, of a cell amenable to maintenance or growth in vitro and includes numerous media useful for maintaining or growing a variety of prokaryotic or eukaryotic cells.

“Media,” and “medium,” are given their plain meanings of compositions containing compounds required for the maintenance and/or growth of at least one cell type. For example, a medium may contain an energy source, nutrients, growth factors, and the like, as would be known in the art. These terms are used throughout this application without strict adherence to number and, accordingly, may be used as synonyms, as would be apparent to one of skill from the context of a particular recitation.

“VSL#3” is given the meaning it has acquired in the art of a group of gram-positive bacterial species collectively known and marketed as a probiotic.

“Heat shock protein,” as used herein, refers to any one of a group of proteins known in the art to exhibit a detectable increase in activity, typically reflective of an increase in expression, upon exposure to a thermal stress in at least one cell type.

“Stabilizing IκB” means the act of preserving an IκB protein for a physiologically significant period of time, without regard to whether the protein being stabilized is unmodified or modified, for example by phosphorylation.

“NF-κB activation” means that intracellular NF-κB exhibits an increased level of at least one activity characteristic of this protein, as would be known in the art. Such activation may result from a decreased rate of destruction of NF-κB, an increased rate of production (e.g., expression) of NF-κB, or a combination thereof.

“Chymotrypsin-like” proteasome activity means a protease activity exhibiting at least one characteristic in common with chymotrypsin, such as the common recognition of a cleavage site or structurally related cleavage sites, as would be known in the art.

“Pharmaceutically acceptable excipient,” is a phrase given its plain meaning of a substantially inert substance admixable with a pharmaceutical or bioactive agent as a vehicle to provide a consistency or form suitable for pharmaceutical administration. Such vehicles typically do not produce an allergic or similar untoward reaction when administered to a human.

“MCP-1 release” refers to the separation of Monocyte Chemoattractant Protein-1 from a cell that had produced or harbored it, such as by secretion, as would be known in the art.

“Modulator” means a substance that affects a detectable activity (e.g., of a protein) or process (e.g., a physiological process such as MCP-1 release), regardless of whether the effect is one of promotion (e.g., enhancement) or inhibition.

In view of these definitions, it will be appreciated that the compounds of the invention provide therapies for the treatment of inflammatory disorders, such as IBD, that are superior to those currently available in the art. In one embodiment, the invention provides a composition comprising an isolated, anti-inflammatory, cytoprotective compound derivable from a probiotic-conditioned medium. In addition, the invention provides methods for treating a patient with an inflammatory disorder comprising administering to the patient an isolated, anti-inflammatory, cytoprotective compound derivable from a probiotic-conditioned medium. In other aspects, the invention provides methods for isolating and characterizing at least one compound from a probiotic-conditioned medium that has anti-inflammatory and/or cytoprotective properties, and preferably both types of properties.

The invention provides methods of identifying and characterizing compounds derivable from cell cultures, such as bacterial cultures, that have anti-inflammatory and cytoprotective properties. The invention provides isolated, anti-inflammatory, cytoprotective compounds derivable from probiotic organisms. The invention also provides compositions and methods useful in treating, and/or preventing, inflammatory diseases, particularly inflammatory disease of an epithelium.

I. Isolation of Anti-Inflammatory and Cytoprotective Compounds

Any bacterial strain or probiotic formulation may be screened for anti-inflammatory and cytoprotective compounds. Preferably, the bacteria are non-pathogenic, enteric bacteria. In the specific embodiments disclosed herein, the probiotic formulation, VSL#3 (VSL Pharmaceuticals, Gaithersburg, Md.), was used. This formulation contains Streptococcus salivarius subsp. thermophilus, Lactobacillus casei, Lactobacillus plantarum, Lactobacillus acidophilus, Lactobacillus delbrueckii subsp. bulgaricus, Bifidobacteria longum, Bifidobacteria infantis, and Bifidobacteria breve.

Methods of bacterial cell culture are well known to those of skill in the art. In a preferred method, VSL#3 is cultured in mammalian tissue culture medium. VSL#3 grows readily in mammalian tissue culture medium (e.g., RPMI 1640 or DMEM) under aerobic conditions. Growth in tissue culture medium makes the isolation of secreted factors much more straightforward than if a complex broth is used.

The anti-inflammatory, cytoprotective compounds of the invention are soluble factors derivable from a cell-conditioned medium such as a VSL#3-conditioned medium. To facilitate the identification and characterization of these compounds it is preferable to remove the bacterial cells from the medium. One of skill in the art would be familiar with methods of separating cells from the soluble factors in the medium. For example, the cells may be removed by centrifugation, filtration or a combination of both. In a preferred embodiment, overnight VSL#3 cultures grown at 37° C. in tissue culture medium (e.g., RPMI 1640) are prepared and then centrifuged at 10,000 g for 5 min at 4° C. The medium is then removed and filtered through a 0.2 μm cellulose acetate filter to exclude all live and intact bacteria. This “conditioned medium” is then used as the source from which anti-inflammatory and cytoprotective compounds are identified.

A. Organic Extraction

The anti-inflammatory and cytoprotective compounds can be further isolated from the conditioned medium by extraction with organic solvents. Organic extraction separates organic from aqueous compounds. Methods of extraction and suitable organic solvents are well known to those of skill in the art. In a preferred embodiment, the organic extraction is performed with ether. The ether extraction process generally removes organic acids and their derivatives, as well as lipid and phospholipid molecules, whereas inorganic salts, hydrophilic peptides, hydrophilic proteins, carbohydrates and polysaccharides tend to remain in the aqueous phase.

The anti-inflammatory and cytoprotective compounds of the invention are present in the ether-extracted fraction and many, if not all, have a molecular weight of less than 10 kDa. It is expected that an anti-inflammatory and cytoprotective compound of the invention is an organic acid.

B. Thin Layer Chromatography

Methods for the purification of organic acids are well known to those of skill in the art. For example, the compounds of the invention may be purified from the ether-extracted fraction of the conditioned medium using thin layer chromatography (TLC), which is a chromatographic technique that is useful for separating organic compounds such as organic acids and their derivatives.

In a preferred embodiment, ether extracts of VSL#3-conditioned medium will be subjected to thin layer chromatography (TLC) on a silica gel G TLC plate that has been activated at 150° C. for 6 hours. The plate will be developed using ethanol/ammonia/water in a ratio of 50:8:6 by volume for the first dimension, and benzene/methanol/acetic acid in a ratio of 45:8:4 for the second dimension. Because of differences in their partitioning behaviors between the mobile liquid phase and the stationary phase, the different components in the ether-extracted mixture will migrate at different rates, allowing for their separation. The chromatogram will then be developed reversibly under iodine vapor, which binds to carbon double bonds and allows visualization of the individual components of the ether-extracted mixture. The separated components will be individually isolated by scraping the visualized spots off with a spatula, allowing the iodine vapor to evaporate, and then back extracting again with ether. Each fraction will then be tested for activity. To ensure that the ether extraction process itself is not exerting an effect on bioactivity, conditioned medium from the DH5α laboratory strain of E. coli will be treated in the same manner as above and used as a negative control for the ether extraction process.

C. Other Separation Techniques

Other separation techniques known to those of skill in the art may also be employed in the invention to fractionate the conditioned medium. High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, and the like. There also is virtually no adsorption, less zone spreading and the elution volume is related in a simple matter to molecular weight.

Separation techniques based on charge may also be used. One such technique is ion exchange chromatography. With ion exchange chromatography, the sample is reversibly bound to a charged matrix. Matrices containing diethyl aminoethyl (DEAE) and carboxymethyl (CM) celluloses are commonly used. Desorption is then brought about by increasing the salt concentration or by altering the pH of the mobile phase. Another technique known to those skilled in the art for separating compounds based on charge is IEF (isoelectric focusing).

Additionally, the conditioned medium may be passed through filters with specific molecular weight cutoffs. For example, some fractions of the invention were parsed by passage through Centricon filters with a 10 kDa molecular weight cutoff.

During the course of purification or isolation, it may be desirable to assay the fractions in order to follow those fractions that retain anti-inflammatory and cytoprotective activity. For example, the medium or fraction may be screened for the ability to induce cytoprotective heat shock proteins, inhibit NF-κB activity, and inhibit proteasomal function of intestinal epithelial cultured cells. These assays are described in more detail below. Preparations that have biological activity may be frozen in aliquots to be used later for identification, purification, and future production of anti-inflammatory and cytoprotective compounds.

D. Chemical Synthesis

In addition to isolating the anti-inflammatory, cytoprotective compounds of the invention from probiotic-conditioned medium, it is also envisioned that these compounds may be created by chemical synthesis. Methods of chemical synthesis are well known to those of skill in the art.

II. Identification of Anti-Inflammatory and Cytoprotective Compounds

The anti-inflammatory and cytoprotective compounds of the invention may be identified by methods known to those of skill in the art Two preferred methods of identifying the compounds of the invention are preparative TLC (similar to analytical TLC described above but on a larger scale) and HPLC (high performance liquid chromatography).

HPLC will be run using a C8 reversed-phase (RP) column with potassium phosphate buffer (pH2.8)/methanol (95:5) to isolate each compound. Separation of components occurs through hydrophobic interactions with the stationary phase (C8 column), and the mobile phase consisting of an aqueous acidic solution followed by an organic solvent then allows for elution of individual compounds in the mixture. Each compound will be retained on the column until the appropriate concentration of organic solvent displaces it from the C8 stationary phase. Each separated peak will then be collected, and the identification of the eluted compounds will be carried out by using suitable techniques known in the art, such as nuclear magnetic resonance imaging (NMR) and infrared spectroscopy (IR).

Exemplifying the identification of compound(s) using the general technique of HPLC, a RP-HPLC fractionation of VSL#3-conditioned medium was performed using a C18 reverse-phase (RP) analytical column (3.9 mm×300 mm). The mobile phase contained buffer A (0.1% trifluoroacetic acid, i.e., 10 mM TFA) and buffer B (60% acetonitrile in 0.1% TFA), with filtering and degassing of buffers before use. The injection volume was 200 μl of conditioned-medium supernatant, the flow rate was 1.0 ml/minute and the chromatography was performed at room temperature. The elution profile was: 5% Buffer B for 5 minutes, 5% Buffer B to 100% Buffer B for 60 minutes, 100% Buffer B for 10 minutes, and 100% Buffer B to 5% Buffer B for 5 minutes. Detection was at 214 nm and 280 nm (UV) and fractions were collected, frozen at −80° C. and lyophilized. Lyophilized fractions were subsequently dissolved in a suitable solvent (e.g., a buffer compatible with cell viability), as would be known in the art. Fractions showing biological activity were used to identify the bioactive agent(s). Use of RP-HPLC is particularly suitable for identification and/or isolation of a bioactive agent that is an organic acid or other relatively non-polar compound, as would be recognized in the art.

In some embodiments, the compounds of the invention may be identified using mass spectrometry. Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). Mass spectrometric methods are well-known to those of skill in the art, and are routinely used for the analysis and characterization of a variety of molecules.

III. Characterization of Anti-Inflammatory and Cytoprotective Compounds

Compounds derivable from probiotic-conditioned medium, such as compounds actually derived therefrom, can be assayed for the ability to induce cytoprotective heat shock proteins, inhibit NF-κB activity, and inhibit proteasomal function of cells such as intestinal epithelial cells.

A. Heat Shock Proteins

Heat shock proteins are a family of proteins that protect a cell against environmental stressors. VSL#3-conditioned medium induces the expression of heat shock proteins, specifically Hsp72 and Hsp25. Hsp72 binds and stabilizes critical cellular proteins, preventing their denaturation. It also has anti-apoptotic actions through preservation of mitochondrial integrity, inhibition of cytochrome C leakage, and blockade of caspase 8 activity (Liu et al., 2003). Hsp25/27 is an actin-stabilizing agent and preserves cytoskeletal and tight junction functions.

Methods of analyzing the induction of heat shock proteins are known to those of skill in the art. For example, the induction of Hsp72 and Hsp25 can be performed by standard Western blot analysis using monoclonal antibodies specifically recognizing and binding specific Hsp isoforms (Stressgen). Immunoblots for the constitutive heat shock cognates, Hsp60 and Hsc73, can be performed to check the specificity of response and to ensure equal loading of lanes (the expression of these proteins usually remains constant). In addition, antibodies can be used to detect the expression of heat shock proteins by immunofluorescence and ELISA.

Other methods of analyzing the induction of heat shock proteins include assaying Hsp mRNA levels using, for example, RT-PCR, genomic microarrays, and real-time PCR. Another approach for analyzing the induction of heat shock proteins is the use of electrophoretic mobility shift assays, for example to look at binding of the transcription factor HSF-1. In addition, HSE-luciferase reporter assays can be employed to measure activity of the transcription factor HSF-1.

B. The NF-κB Pathway

A number of approaches are known to those of skill in the art to assess the inhibition of NF-κB activation, such as inhibition of the NF-κB pathway. For example, electrophoretic mobility shift assays (EMSA or gel shifts) using an oligonucleotide labeled with ³²P can be performed to determine activation of NF-κB. Activation of NF-κB and release from the inhibitor IκB results in binding to this mimic, which can be easily detected on polyacrylamide gels. At least two additional measures may be used to corroborate NF-κB activation. First, activated NF-κB translocates into the nucleus of the cell and therefore detection of NF-κB in the nucleus by immunofluorescence or immunoblotting of nuclear fractions strongly supports NF-κB activation. Second, transient transfections with a NF-κB-sensitive reporter construct, such as a construct having five copies of the NF-κB responsive promoter element cloned in front of a firefly luciferase reporter, can be performed. Moreover, data from the three assays (EMSA, nuclear NF-κB translocation, and NF-κB reporter) may help identify unique steps at which the compounds of the invention modulate, e.g., inhibit, NF-κB activity.

ELISA-based assays for the detection of NF-κB activation are also known in the art. For example, an NF-κB ELISA-based assay kit is commercially available from Vinci-Biochem (Vinci, Italy).

NF-κB regulates a wide variety of genes encoding, for example, cytokines, cytokine receptors, cell adhesion molecules, proteins involved in coagulation, and proteins involved in cell growth. Thus, another approach to the study of the NF-κB pathway is through the analysis of the expression of genes known to be regulated by NF-κB. Those of skill in the art will be familiar with a variety of techniques for the analysis of gene expression. For example, changes in mRNA and/or protein levels may be measured. Changes in mRNA levels can be detected by numerous methods including, but not limited to, real-time PCR and genomic microarrays. Changes in protein levels may be analyzed by a variety of immuno-detection methods known in the art.

It is also worthwhile to monitor changes in the NF-κB regulator, IκB. As the compounds of the invention are expected to affect the activity of IκB in more than one form, antibodies to both the native as well as the phosphorylated form of IκB are useful and may be used for Western blotting and immunohistochemical localization.

C. The Proteasome

Finally, the compounds of the invention may be screened by assessing their effects on cellular proteasomal function. The proteasome is a large complex, which contains several protease activities with different specificities. It exists in two forms, a 20S complex and a 26S complex. Cellular proteasomes play an important role in degrading cellular proteins as well as in providing viral and endogenous peptide fragments for loading of MHC I molecules for antigen presentation.

Inhibitors of the proteasome block the degradation of many cellular proteins. Proteasome inhibitors are broadly categorized into two groups: synthetic analogs and natural products. Synthetic inhibitors are peptide-based compounds with diverse pharmacophores. These include peptide benzamides, peptide α-ketoamides, peptide aldehydes, peptide α-ketoaldehydes, peptide vinyl sulfones, and peptide boronic acids. Known natural product proteasome inhibitors include linear peptide epoxyketones, peptide macrocycles, γ-lactam thiol ester, and epipolythiodioxopiperazine toxin. Some specific examples of proteasome inhibitors include MG132, ALLN, E64d, LLM, quinacrine, chloroquine, clioquinol, (R)-(−)-3-hydroxybutyrate, dopamine, L-DOPA, PR39, gliotoxin, and green tea (EGCG). Additional examples of proteasome inhibitors are disclosed in Kisselev and Goldberg (2001) and Myung et al. (2001), both of which are incorporated herein by reference in their entireties.

Inhibition of proteasomal function by VSL#3-conditioned medium provides a potential unifying mechanism for the inhibition of NF-κB and induction of cytoprotective heat shock proteins. Such an action is consistent with the accumulation of phospho- and ubiquitinated-IκB disclosed herein. Furthermore, it has been shown that inhibition of proteasomal function is an extremely potent stimulus of the heat shock protein response, likely due to the accumulation of undegraded proteins (Lee and Goldberg, 1998). Although not wishing to be bound by theory, the data disclosed herein indicate that the primary mechanism of action through which VSL#3-conditioned medium inhibits the NF-κB pathway and induces Hsp expression appears to be direct inhibition of proteasomal function. This represents a novel mechanism of probiotic action differing from that reported by Neish, et al. who had reported inhibition of activated NF-κB by non-pathogenic Salmonella organisms through a type III secretion system, which requires intact bacteria and bacterial adherence.

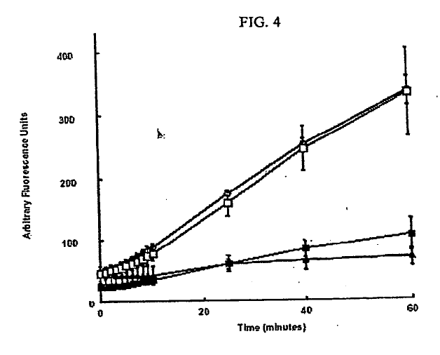

Those of skill in the art are familiar with methods for assaying proteasome function. In a preferred method, proteasome assays are performed using a fluorometric assay by preparing crude cell lysates from YAMC cells treated with VSL#3-conditioned medium, then adding the proteasome substrate SLLVY-AMC and measuring hydrolysis of this product over time (see FIG. 4). The substrate is a five amino acid peptide attached to a fluor (4-amino-7-methylcoumarin) which, upon cleavage by the chymotrypsin-like activity of the proteasome, results in a fluorescent signal that can be measured and plotted over time. The activity of the proteasome is reflected by the rate, or slope of the line. In this assay, the inhibition of proteasome activity by a candidate molecule may be compared to that of a known proteasome inhibitor, such as MG132.

Another method for assaying proteasome function is immunofluorescence using antibodies that recognize active proteasomes. For example, LMP2 antibodies specifically recognize the proteasome beta subunit. In addition, proteasome assay kits are commercially available from Biomol International LP.

D. Animal Models

The characterization of the compounds of the invention may involve the use of various animal models, including transgenic animals that have been engineered to have specific defects, or to carry markers that can be used to measure the ability of a candidate substance to reach and affect different cells within the organism. Due to their size, ease of handling, and information on their physiology and genetic make-up, mice are a preferred animal model, especially for transgenics. However, other animals are suitable as well, including rats, rabbits, hamsters, guinea pigs, gerbils, woodchucks, cats, dogs, sheep, goats, pigs, cows, horses, and monkeys (including chimps, gibbons and baboons). Assays may be conducted using an animal model derived from any of these species:

Some examples of mouse models for colitis include the DSS-induced colitis model, IL-10 knockout mouse, A20 knockout mouse, TNBS-induced colitis model, IL-2 knockout mouse, TCRalpha receptor knockout mouse, and the E-cadherin knockout mouse.

Treatment of animals with test compounds will involve the administration of the compound, in an appropriate form, to the animal. Any animal model of inflammatory disease known to those of skill in the art can be used in the practice of a method according to the invention. Administration will be by any route that could be utilized for clinical or non-clinical purposes. For example, the compound may be delivered by gavage or by rectal administration. In addition, the protective effects of a compound may be assayed by administering a compound before inducing colitis in the animal model. Alternatively, the therapeutic effect of a compound may be assayed by administering the compound after inducing colitis in the animal model.

Determining the effectiveness of a compound in vivo may involve consideration of a variety of different criteria. One of ordinary skill in the art would be familiar with the wide range of techniques available for assaying for inflammation in a subject, whether that subject is an animal or a human subject. For example, inflammation can be measured by histological assessment and grading of the severity of colitis. Other methods for assaying inflammation in a subject include, for example, measuring myeloperoxidase (MPO) activity, transport activity, villin expression, and transcutaneous electrical resistance (TER) or transepithelial electrical resistance (TEER).

The effectiveness of a compound can also be assayed using tests that assess cell proliferation. For example, cell proliferation may be assayed by measuring 5-bromo-2′-deoxyuridine (BrdU) uptake. Yet another approach to determining the effectiveness of a compound would be to assess the degree of apoptosis. Methods for studying apoptosis are well known in the art and include, for example, the TUNEL assay.

In addition, measuring toxicity and dose response can be performed in animals rather than in in vitro or in cyto assays.

IV. Pharmaceutical Compositions

Compositions of the invention comprise an effective amount of an anti-inflammatory, cytoprotective compound, which may be dissolved and/or dispersed in a pharmaceutically acceptable excipient, such as a carrier and/or aqueous medium.

The anti-inflammatory, cytoprotective compounds of the invention may be delivered by any method known to those of skill in the art (see for example, “Remington's Pharmaceutical Sciences” 15th Edition). For example, the pharmaceutical compositions may be delivered orally, rectally, parenterally, or topically.

Solutions comprising the compounds of the invention may be prepared in water suitably mixed with a surfactant, such as polyethylene glycol (PEG) of low (less than 8 kDa) or high (greater than 8, and preferably greater than 15, kDa) average molecular weight, or hydroxypropylcellulose. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. The form should usually be sterile and must be fluid to the extent that effective syringability exists. It must be stable under the conditions of manufacture and storage and must be preserved against the contaminating action of microorganisms, such as bacteria and fungi.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral, and intraperitoneal administration. In this connection, sterile aqueous media that can be employed will be known to those of skill in the art in light of the present disclosure.

A suppository may also be used. Suppositories are solid dosage forms of various weights and/or shapes for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%. The pharmaceutical compositions of the invention may also be delivered by enema.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained-release formulations and/or powders. In certain defined embodiments, oral pharmaceutical compositions will comprise an inert diluent and/or assimilable edible carrier, and/or they may be enclosed in hard- and/or soft-shell gelatin capsule, and/or they may be compressed into tablets, and/or they may be incorporated directly with the food of the diet. For oral therapeutic administration, the active compound(s) may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, such as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose, as a sweetening agent, methyl and/or propylparabens as preservatives, and a dye and/or flavoring, such as cherry and/or orange flavor.

Topical formulations include, creams, ointments, jellies, gels, epidermal solutions or suspensions, and the like, containing the active compound.

For human administration, preparations should meet sterility, pyrogenicity, general safety and purity standards as required by the FDA Office of Biologics standards.

The dosage of the anti-inflammatory, cytoprotective compounds and dosage schedule may be varied on a subject-by-subject basis, talking into account, for example, factors such as the weight and age of the subject, the type of disease being treated, the severity of the disease condition, previous or concurrent therapeutic interventions, the manner of administration, and the like, which can be readily determined by one of ordinary skill in the art.

Administration is in any manner compatible with the dosage formulation, and in such amount as will be therapeutically effective. The quantity to be administered depends on the subject to be treated. Precise amounts of an active ingredient required to be administered depend on the judgment of the practitioner and such judgments may involve routine procedures to determine an effective amount on a case-by-case basis.

The following examples are included to demonstrate preferred embodiments of the invention. It will be appreciated by those of skill in the art that the techniques disclosed in the examples which follow represent techniques disclosed herein as functioning well in the practice of the invention However, those of skill in the art will, in light of the present disclosure, appreciate that many changes can be made in the specific embodiments which are disclosed and still obtain a like or similar result without departing from the spirit and scope of the invention. In brief, the following examples illustrate various embodiments of the invention: Example 1 describes basic techniques used in the work disclosed herein, including tissue culture and cell lysate preparation, NF-κB, ⁵¹Cr, G/F actin and proteasome activity assays, electrophoretic mobility shift assays, Western blot analysis of proteins, MCP-1 assays, and statistical analyses of the observed data; Example 2 discloses the inhibition of NF-κB activity by probiotic-conditioned medium; Example 3 describes the inhibition of Monocyte Chemoattractant Protein-1 (MCP-1) release by probiotic-conditioned medium; Example 4 describes the inhibition of IκB degradation (including phosphorylated IκB) by probiotic-conditioned medium; Example 5 describes the failure of probiotic-conditioned medium to universally inhibit protein ubiquitination; Example 6 shows that probiotic-conditioned medium inhibits proteasome activity, and does so more rapidly than protein expression is induced; Example 7 addresses the induction of heat shock protein expression by probiotic-conditioned medium, including the time course of such induction, and provides evidence that the induction is mediated by HSF-1 induction; Example 7 describes the properties of bioactive probiotic agents (i.e., anti-inflammatory, cytoprotective compounds); Example 8 discloses that probiotic-conditioned medium protects epithelial cells from oxidant stress; Example 9 discloses some properties of bioactive probiotic compounds or agents; and Example 10 establishes that probiotic agents differentially inhibit proteasome activities.

### EXAMPLE 1

General Methodologies

### Probiotic Bacterial Culture and Generation of Conditioned Media

The probiotic formulation, VSL#3 (VSL Pharmaceuticals, Gaithersburg, Md.), contains Streptococcus salivarius subsp. thermophilus, Lactobacillus casei, Lactobacillus plantarum, Lactobacillus acidophilus, Lactobacillus delbrueckii subsp. bulgaricus, Bifidobacteria longum, Bifidobacteria infantis, and Bifidobacteria breve at a concentration of 5×10¹¹ lyophilized bacteria/gram. VSL#3 (batch number 2034-A2, VSL Pharmaceuticals, Gaithersburg, Md.) was grown to a concentration of approximately 2×10¹⁴ (as determined by colony counts) in phenol red-free RPMI 1640 medium for 16 hours, then centrifuged at low speed in a tabletop Sorvall centrifuge (3000×g, 4° C., 10 minutes). The supernatant (conditioned medium) was then passed through a 0.22 micron low protein-binding filter (Millipore, Bedford, Mass.) to remove all bacterial cells and debris. Aliquots of conditioned medium were stored in sterile microcentrifuge tubes at −80° C. until further use.

Tissue Culture

YAMC (young adult mouse colon) cells are a conditionally immortalized mouse colonic intestinal epithelial cell line derived from the Immortimouse that express a transgene of a temperature-sensitive SV40 large T antigen (tsA58) under control of an interferon-gamma-sensitive portion of the MHC class II promoter (Whitehead et al, 1993). YAMC cells were maintained under permissive conditions (33° C.) in RPMI 1640 medium with 5% (vol/vol) fetal bovine serum, 5 U/ml murine IFN-γ (GibcoBRL, Grand Island, N.Y.), 50 μg/ml streptomycin, 50 U/ml penicillin, supplemented with ITS+ Premix (BD Biosciences, Bedford, Mass.). Under non-permissive (non-transformed) conditions at 37° C. in the absence of IFN-γ, these cells undergo differentiation and develop mature epithelial cell functions and properties including tight junction formation, polarity, microvillar apical membranes, and transport functions.

Cells were plated at a density of 2×10⁵ per 60 mm tissue culture dish (for Western blot analysis and proteasome assays), or at 1×10⁵ per well in 6-well plates (for NF-κB luciferase transfection experiments). After 24 hours of growth at 33° C. to allow for cell attachment, the medium was replaced with IFN-free medium and cells were moved to 37° C. (non-permissive conditions) for 24 hours to allow the development of the differentiated colonocyte phenotype for all experiments. Cells were treated with VSL#3-conditioned medium (1:10 dilution) overnight, and then used the following day in various experiments. For NF-κB luciferase reporter assays, murine TNF-α (Peprotech, Rocky Hill, N.J.) at a concentration of 50 ng/ml was added directly to the cells at this time and left for 6 hours before harvest. Heat shock controls were exposed to 42° C. for 23 minutes and allowed to recover at 37° C. for 2 hours before harvest. MG132-treated control cells were treated for 2 hours with 25 μM MG132 (Biomol, Plymouth Mtg, Pa.) at 37° C. prior to harvest unless otherwise specified.

Two other cell lines, MSIE (a small intestine YAMC counterpart cell line) and 3T3 fibroblasts, were used in this study and maintained as previously described (Kojima, 2003), incorporated herein by reference.

Preparation of Cell Lysates

Cells were washed twice and then scraped in ice-cold HBS (150 mM NaCl, 5 mM KCl, 10 mM HEPES, pH 7.4). Cells were pelleted (14,000×g for 20 seconds at room temperature), then resuspended in ice-cold lysis buffer (10 mM Tris, pH 7.4, 5 mM MgCl₂, 50 U/ml DNAse and RNAse, plus complete protease inhibitor cocktail (Roche Molecular Biochemicals, Indianapolis, Ind.)). Protein concentrations were determined using the bicinchoninic acid procedure (Smith, 1985). For proteasome assays, samples were stored immediately at −80° C. until use. For Western blots, samples were heated to 75° C. for 5 minutes after addition of 3× Laemmli Stop buffer, then stored at −80° C. until use.

Western Blot Analysis

Twenty micrograms of protein per lane were resolved on 12.5% SDS-PAGE and transferred in 1× Towbin buffer (composition 25 mM Tris, 192 mM glycine, pH 8.8, 15% vol/vol methanol) onto PVDF membranes (Polyscreen, Perkin-Elmer NEN, Boston, Mass.) as previously described (Kojima, 2003), incorporated herein by reference. Membranes were blocked in 5% (wt/vol) non-fat milk in TBS-Tween (Tris-buffered saline (150 mM NaCl, 5 mM KCl, 10 mM Tris, pH 7.4) with 0.05% (vol/vol) Tween 20) for one hour at room temperature. For anti-ubiquitin blots, membranes were blocked in 3% bovine serum albumin (Fisher, Pittsburgh, Pa.). Primary antibody was added to TBS-Tween and incubated overnight at 4° C. with a specific anti-Hsp 25 antibody (SPA 801, Stressgen, Victoria, BC, Canada), anti-Hsp 72 antibody (SPA 810, Stressgen), anti-Hsc 73 antibody (SPA 815, Stressgen), anti-IκB-α antibody (sc-1643, Santa Cruz Biotechnology, Santa Cruz, Calif.), anti-phospho IκB-α antibody (sc-8404, Santa Cruz), or anti-ubiquitin antibody (PW 8810, Affiniti Research Products Ltd, Exeter, U.K.). Blots were then washed in TBS-Tween five times for 10 minutes each at room temperature before incubation with peroxidase-conjugated secondary antibodies (Jackson Immunoresearch Labs, Inc. Fort Washington, Pa.) for 1 hour at room temperature. Membranes were then washed (five times, 10 minutes each) in TBS-Tween followed by a final wash in TBS (no Tween). Blots were visualized with an enhanced chemiluminescence system ECL reagent (Supersignal, Pierce, Rockford, Ill.) and developed as per the manufacturer's instructions.

Statistical Analysis

The luciferase assays were performed in triplicate and the proteasome assays were performed in duplicate for each experiment. All experiments were repeated a minimum of three to six times each. All numerical values are expressed as mean+/−standard error of the mean unless otherwise indicated. Where multiple comparisons were made, ANOVA analysis using a Bonferroni's correction was used to assess significance of differences between groups. P<0.05 was considered statistically significant.

### EXAMPLE 2

### Probiotics Inhibit NF-κB Activation in Intestinal Epithelial Cells

To determine whether the bacteria in VSL#3 secrete factors possessing anti-inflammatory activity, the effects of VSL#3-conditioned media (VSL#3-CM) on the NF-κB pathway were investigated. The ability of VSL#3-CM to block transcriptional activity of NF-κB in intact epithelial cells stimulated by TNF-α was tested using an NF-κB luciferase reporter assay.

NF-κB luciferase assays were performed using the Promega Dual Luciferase Reporter 1000 Assay System (Promega, Madison, Wis.) and plasmids were transfected using TransIT LT-1 transfection reagent (Mirus, Madison Wis.) as per the manufacturer's instructions. Briefly, 2 μg of NF-κB response element-driven firefly luciferase reporter plasmid (Clontech, Palo Alto, Calif.) and 0.2 μg Thymidine Kinase (TK) promoter-driven Renilla reporter plasmid (Promega, Madison Wis.) were mixed with LT-1 polyamine transfection reagent. After formation of complexes, the solution was added to YAMC cells at 33° C. and allowed to incubate overnight. Cells were then placed at 37° C. in IFN-γ-free medium. After cells were grown in non-permissive (non-transformed) conditions, VSL#3-conditioned medium (VSL#3-CM) was added to each well at a dilution of 1:10 and left overnight, unless otherwise specified. Murine TNF-α was added at 50 ng/ml the next morning and cells were harvested 6 hours later. NF-κB luciferase assays were performed as per the manufacturer's instructions and luminescence measured in a Berthold Luminometer (Oakridge, Tenn.). Co-transfection with TK-Renilla, which displays constitutive low levels of activity, is used as an internal control against which to normalize the NF-κB luciferase data. Experiments were performed in triplicate.

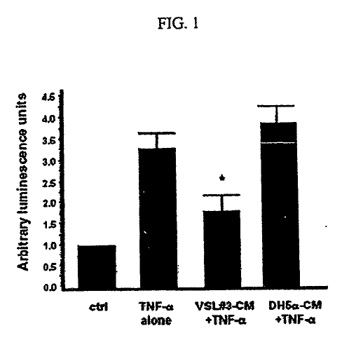

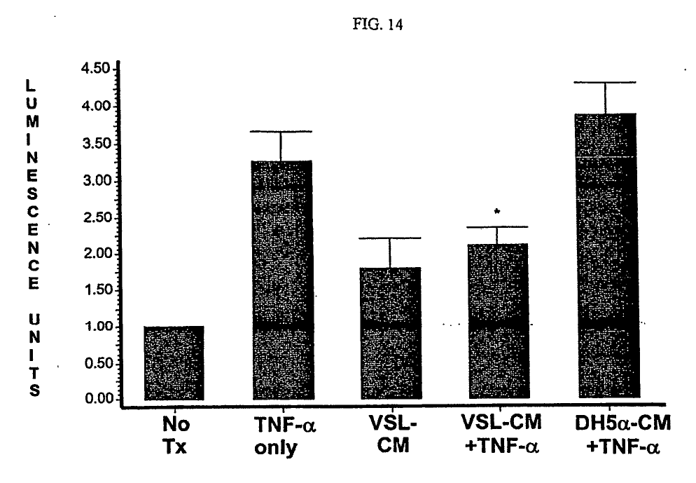

Young adult mouse colon cells transiently transfected with the reporter gene expressed a low level of baseline NF-κB activity, which increased upon stimulation with TNF-α, as reflected by an increase in luciferase activity (FIGS. 1 and 14). Pretreatment with VSL#3-CM for 16 hours resulted in the attenuation of TNF-α-induced NF-κB activity in epithelial cells by 45% compared to TNF-α treatment alone (FIG. 14, 1.80+/−0.41 for VSL#3-CM-treated cells vs. 3.25+/−0.41 with TNF-α alone, p<0.05). This effect was specific to VSL#3-CM, as pretreatment of epithelial cells with conditioned medium from the E. coli strain DH5α did not attenuate TNF-α-induced NF-κB activation (FIG. 1, column 4; FIG. 14, column 5). Although not wishing to be bound by any theoretical implications of studies into the mechanism(s) underlying the influence of VSL#3-conditioned medium on TNF-α-induced NF-κB activation, both electrophoretic mobility shift assay and ELISA analyses did not show a significant impact of VSL#3-CM on the binding of nuclear NF-κB (p65 subunit).

### EXAMPLE 3

### Probiotic-conditioned Medium Inhibits MCP-1 Release

Probiotics decrease release of Monocyte Chemoattractant Protein 1 (MCP-1) in response to NF-κB stimulation by TNF-α. MCP-1 is an endogenous immune response gene that has been implicated in the pathogenesis of many inflammatory diseases such as multiple sclerosis, rheumatoid arthritis, and IBD. Like IL-8, studies have shown that MCP-1 is highly expressed in areas of active inflammation in Crohn's disease and its expression depends on NF-κB activation.

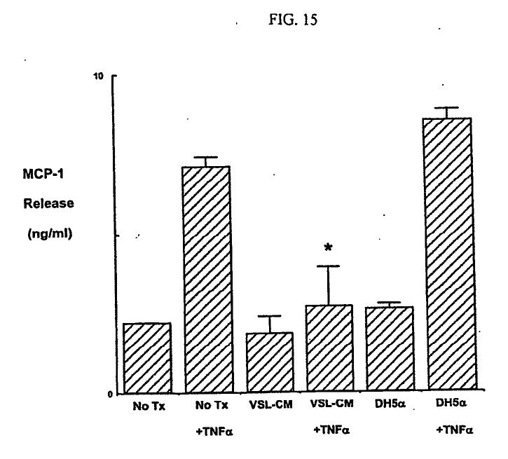

YAMC cells were grown and treated with VSL#3-CM and subsequently treated with TNF-α, as described herein, to stimulate NF-κB activation. Supernatants were harvested and tested for the production of MCP-1 using a mouse MCP-1 ELISA kit (Pierce Endogen, Rockford, Ill.) as per the manufacturer's instructions to measure MCP-1 release from the cells. Treatment of intestinal epithelial cells with VSL#3-CM attenuated the release of MCP-1 in response to NF-κB stimulation by TNF-α (FIG. 15, column 4). No significant difference in MCP-1 release was noted in cells treated with VSL#3-CM alone compared to untreated control cells (compare FIG. 15, columns 1 and 3).

Consistent with the results of Example 2, demonstration that VSL#3-CM inhibits release of MCP-1 establishes a role for an isolated, anti-inflammatory compound derivable from VSL#3-CM in the prevention and/or treatment of inflammatory disorders, such as IBD (e.g., Crohn's disease, ulcerative colitis).

### EXAMPLE 4

### Probiotics Inhibit Degradation of the NF-κB Inhibitor IκB in Intestinal Epithelial Cells

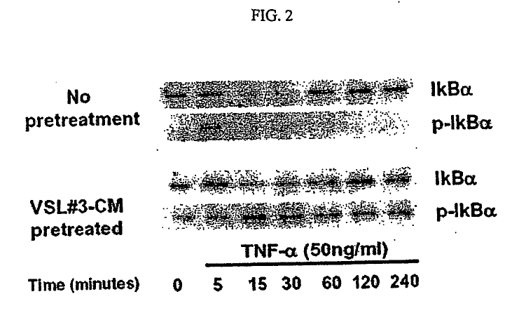

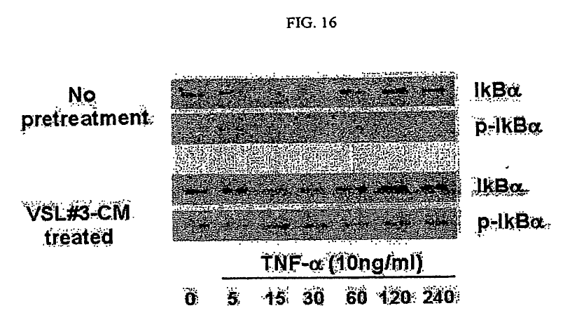

To determine which step(s) in the pathway of NF-κB activation is the step(s) at which VSL#3-CM exerts its effects, the regulation of the NF-κB inhibitory molecule, IκBα, was investigated. The effects of VSL#3-CM on total IκBα and phosphorylated IκBα protein in YAMC cells treated with TNF-α were examined. Pretreatment with VSL#3-CM inhibits degradation of the phosphorylated form of IκBα in TNF-α-treated cells (FIG. 16, bottom two panels or rows). In the absence of VSL#3 treatment, TNF-α stimulates phosphorylation of IκBα within 5 minutes, followed by rapid degradation of IκBα at 15 and 30 minutes (FIG. 16, top two panels or rows). Subsequently, NF-κB stimulates IκBα expression, shutting down further NF-κB activation (FIG. 16, see lane on top panel or row at 60 minutes). In contrast, when intestinal epithelial cells are treated with VSL#3-CM prior to TNF-α stimulation, phosphorylated IκBα is stabilized and resists degradation for over 2 hours (FIG. 16, lower bottom panel or row). Furthermore, the amount of total IκBα never declines throughout the period of TNF-α stimulation, thus indicating that VSL#3-CM inhibits pathways of IκBα degradation normally associated with TNF-α stimulation of intestinal (gut) epithelial cells. While VSL#3-CM had some basal yet undefined regulatory effect at the level of IκB phosphorylation as shown in FIG. 16 (lane 1), no effect of VSL#3-CM alone was seen on MCP-1 secretion or release by YAMC cells in the absence of TNF-α as shown in FIG. 2 (column 3).

The results are consistent with a view that VSL#3-CM inhibits NF-κB activation by protecting IκBα (i.e., by inhibiting its degradation), but the data obtained to date has not established a single, integrated mechanism for the effect that VSL#3-CM has on NF-κB activation. The invention, however, is not dependent on any particular mechanism of action and the scope of the appended claims should not be limited by any theoretical consideration of such mechanism(s).

### EXAMPLE 5

### Probiotics do not Inhibit Ubiquitination in Intestinal Epithelial Cells

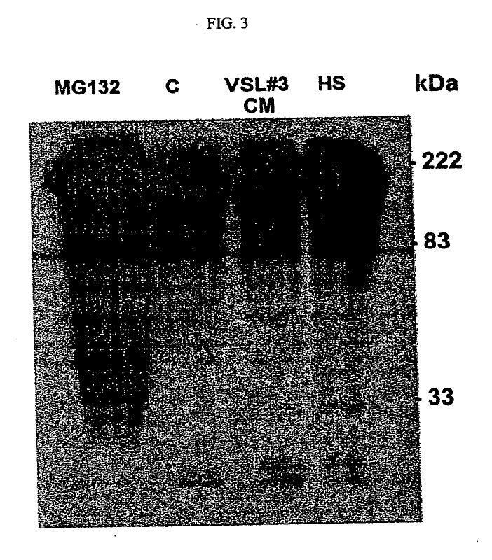

The lack of IκBα degradation in response to TNF-α in VSL#3-CM-treated cells indicates that the probiotic-CM interferes with one or more downstream activation events, namely the steps of ubiquitination and/or proteasomal degradation of IκBα. A nonpathogenic strain of Salmonella typhimurium has been reported to inhibit degradation of IκBα through blockade of ubiquitination (Neish, 2000). These effects of Salmonella typhimurium may represent one method by which gastrointestinal flora are able to modulate the immune system and thus live in symbiosis with a eukaryotic host. To test whether a similar mechanism is involved in the inhibition of NF-κB by VSL#3-CM, immunoblot analyses using anti-ubiquitin antibodies were performed on total cell protein from intestinal epithelial cells treated with VSL#3-CM (FIG. 3). In contrast to what might have been expected based on the ability of nonpathogenic S. typhimurium to inhibit epithelial ubiquitin ligase, treatment of epithelial cells with VSL#3-CM does not result in a decrease of ubiquitinated proteins compared to untreated cells. In fact, VSL#3-CM treatment actually results in accumulation of certain ubiquitinated proteins.

One mechanism by which this could occur is through inhibition of proteasome function, which results in accumulation of undegraded, ubiquitinated proteins (Voges, 1999). While not as dramatic as the effects of the proteasome inhibitor MG132, the amount and the pattern of increase in ubiquitinated proteins upon VSL#3-CM treatment is similar to what is seen with thermal stress.

### EXAMPLE 6

### Probiotics Inhibit Proteasome Activity in Intestinal Epithelial Cells

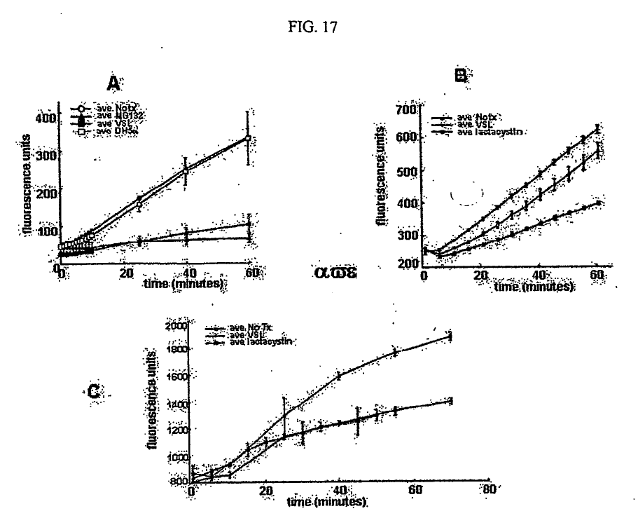

Proteasome inhibitors which block NF-κB activation through inhibition of IκBα degradation have already been described (Gao, 2000). Since probiotic treatment results in both attenuation of NF-κB activity and inhibition of IκBα degradation, the effect of VSL#3-CM on proteasome activity, as measured by cleavage of the SLLVY-AMC substrate, was investigated (FIG. 17).

Proteasome activity from cell lysates was determined using a 20S Proteasome assay kit (Calbiochem, San Diego, Calif.). Briefly, ice-cold cell lysate containing 20 μg protein was added to proteasome assay reaction buffer (25 mm HEPES, 0.5 mM EDTA, pH 7.6) activated with 0.03% (wt/vol) SDS. The sample was allowed to come to room temperature, then placed in a quartz cuvette and 10 μM of the substrate suc-leu-leu-val-tyr-AMC (SLLVY-AMC), Bz-val-gly-arg-AMC, or Z-leu-leu-glu-AMC was added. The SLLVY-AMC substrate is cleaved by the chymotrypsin-like activity of the proteasome, the Bz-val-gly-arg-AMC substrate is cleaved by the trypsin-like activity of the proteasome, and the Z-leu-leu-glu-AMC substrate is cleaved by the PGPH, or caspase-like, activity of the proteasome. Proteasome activity was determined by measuring the fluorogenic signal generated by cleavage of AMC (7-amino-4-methylcoumarin) from the peptide moiety of the proteasome substrate. Fluorescence (excitation at 380 nm, emission at 460 nm) was measured every minute for the first 10 minutes, then every 15 minutes thereafter in a Hitachi F-2000 fluorometer (Hitachi, Japan). Cells were treated with either MG132 (25 μM) or lactacystin (10 μM) as a positive inhibitor control. Untreated cells were treated with DMSO as a vehicle control for MG132 and lactacystin. Experiments were performed in triplicate. For each experiment, all time points were performed in duplicate.

Extracts from cells treated with VSL#3-CM were compared with untreated controls, cells treated with MG132 (a potent proteasome inhibitor), and DH5α (E. coli)-CM for proteasome activity. Epithelial cells treated with VSL#3-CM displayed markedly lower levels of proteasome activity as compared to untreated controls, and inhibition by VSL#3-CM was almost as pronounced as what was seen with the synthetic proteasome inhibitor MG132. This effect is specific to VSL#3, as DH5α-CM (E. coli) does not exert any inhibitory effects on proteasome function.

The modest accumulation of ubiquitinated proteins upon VSL#3-CM treatment relative to the accumulation in response to the known proteasome inhibitor MG132 is consistent with the finding that VSL#3-CM is less toxic than MG132. Accordingly, an isolated, anti-inflammatory compound derivable from VSL#3-CM is expected to be more therapeutically acceptable than such known proteasome inhibitors as MG132.

### a. Time Course of Proteasome Activity Inhibition

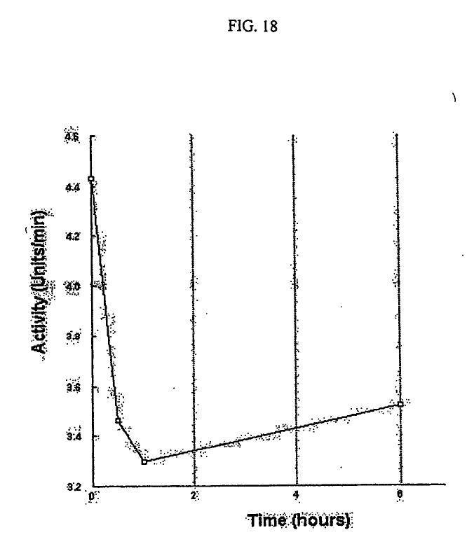

A time course of VSL#3-CM treatment was performed in order to determine the speed with which VSL#3-CM is able to elicit the proteasome inhibition described above. Cells were treated for 30 minutes, 60 minutes, and 6 hours, then harvested and assayed for their ability to inhibit the CTL-like activity of the proteasome (FIG. 18). It was found that the most pronounced proteasome inhibition occurs early after probiotic treatment, with over 50% of the inhibition occurring within the first 30 minutes, consistent with what is reported with other proteasome inhibitors. This indicates that proteasome inhibition by VSL#3-CM is an early event, occurring almost immediately after exposure of the epithelial cells to the probiotic-conditioned medium. This finding, considered in view of the relative lack of toxicity of VSL#3-CM, indicates that an isolated, anti-inflammatory compound derivable from VSL#3-CM would be a safe and quick-acting, i.e., an effective, prophylactic and/or therapeutic for inflammatory disorders such as IBD.

### EXAMPLE 7

### Probiotics Display Differential Inhibition of Proteasome Activities

In addition to its effects on the chymotrypsin-like activity of the proteasome as described herein, VSL#3-CM possesses some weak inhibitory activity against the caspase-like proteolytic function of the proteasome and has no inhibitory effect on its trypsin-like activity.

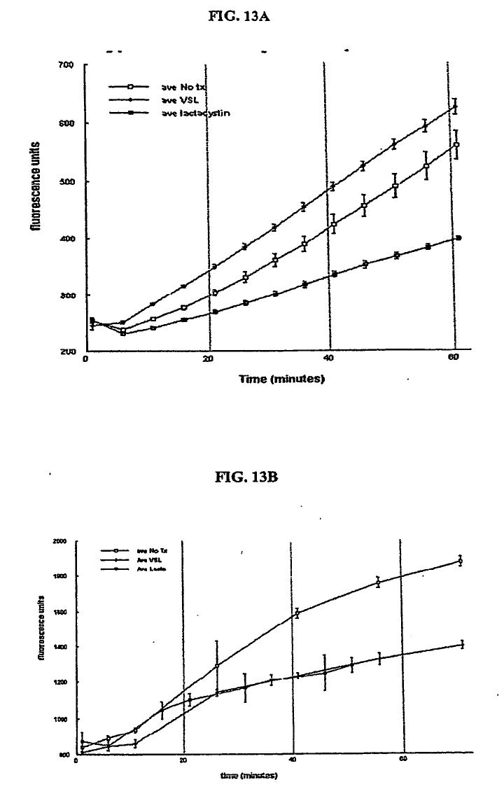

YAMC cells were treated with VSL#3-conditioned medium for 16 hours and then harvested for proteasome assay. Proteasome activity was measured in cell lysates using either the fluorogenic substrate Bz-Val-Gly-Arg-AMC, which measures the trypsin-like protease activity (FIG. 13A), or the substrate Z-Leu-Leu-Glu-AMC, which is cleaved by the PGPH, or caspase-like, activity of the proteasome (FIG. 13B). As a positive inhibitor control, lactacystin was used at a concentration of 10 μM. Lactacystin inhibits both the trypsin-like and chymotrypsin-like activities but is only a weak inhibitor of the caspase-like activity of the proteasome.

The results show that VSL#3-conditioned medium has no inhibitory effect on the trypsin-like activity (FIG. 13A) and has only a weakly inhibitory effect on the caspase-like activity of gut epithelial proteasomes, about equivalent to 10 μM lactacystin (FIG. 13B). This indicates that VSL#3-CM does not globally inhibit all proteolytic functions of the proteasome, but rather displays some specificity or affinity for some functions.

Treatment with VSL#3-CM is well tolerated by the epithelial cells, which is not the case with most of the synthetic proteasome inhibitors. The lower toxicity of VSL#3-CM may be due to its differential affinity, which likely allows some normal functioning of the proteasome to continue while the degradation of certain proteins such as IκB is blocked. Without wishing to be bound by theory, this may in part explain why the pattern of accumulated ubiquitinated proteins is different in cells treated with VSL#3 from what is observed with the more powerful and toxic synthetic inhibitors such as MG132.

### EXAMPLE 8

### Probiotics Induce Heat Shock Proteins in Intestinal Epithelial Cells

It has been shown that enteric flora (luminal bacteria of the colon) in the gut influence expression of epithelial heat shock proteins (Kojima 2003; Beck, 1995). Proteasome inhibitors are potent inducers of heat shock protein expression through activation of the heat shock transcription factor, HSF-1 (Pirkkala, 2000). Accordingly, experiments were conducted to determine whether heat shock protein expression occurred concomitantly with proteasome inhibition.

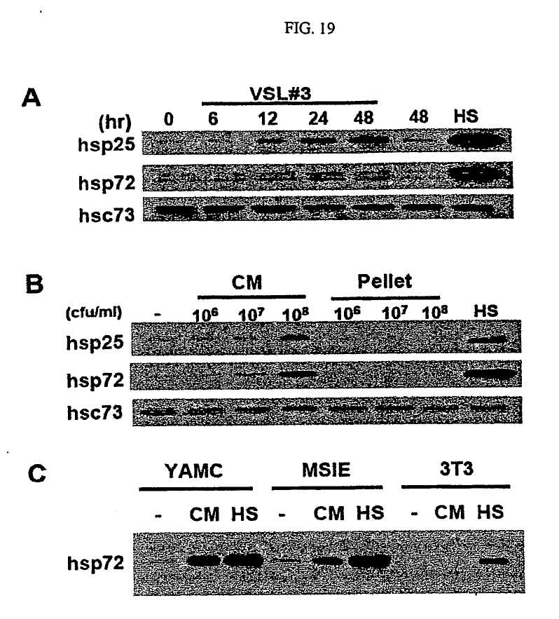

YAMC cells were co-cultured with the probiotic VSL#3 to test its ability to induce heat shock protein expression. By immunoblot analysis, VSL#3 was shown to induce Hsp25 and Hsp72 expression in YAMC cells beginning at 6 to 12 hours (FIG. 19 panel A). Expression of the constitutively expressed heat shock protein Hsc73, serving as a loading control, was not affected by VSL#3. Untreated cells left in culture for 48 hours do not mount an Hsp response, demonstrating that the effect is specific to the probiotic treatment and not a time-dependent characteristic of the cells grown in culture.

Further, YAMC cells were either treated with VSL#3-CM (for times varying from 15 minutes to 6 hours), or heat shocked as described herein. Whole cell extracts were prepared in lysis buffer (25% vol/vol glycerol, 420 mM NaCl, 1.5 mM MgCl₂, 0.2 mM EDTA, 0.5 mM DTT, 20 mM HEPES, pH 7.4, with the Complete Protease Inhibitor Cocktail) by freezing once in a dry ice/alcohol bath, thawing on ice, shearing gently with a pipette tip, and centrifugation at 50,000×g for 5 minutes at 4° C. Cell extract containing ten micrograms protein was mixed with ³²P-labeled HSE oligonucleotide (containing four tandem inverted repeats of the heat shock element (nGAAn): CTAGAAGCTTCTAGAAGCTTCTAG (SEQ ID NO: 1)) and 0.5 μg poly (dI-dC) in binding reaction buffer (final concentrations 20 mM Tris, pH 7.4, 100 mM NaCl, 1 mM EDTA, 10% vol/vol glycerol). The binding reaction was allowed to incubate for 25 minutes at 25° C. and then analyzed on a 4% non-denaturing polyacrylamide gel run in 0.5×TBE buffer. Gels were dried and autoradiographed to detect DNA-protein complexes. For supershift experiments, YAMC cells were incubated with VSL#3-CM for 6 hours before harvest and 1 μg of specific antibody to either HSF-1 (SPA-950, Stressgen, Victoria, BC, Canada) or HSF-2 (sc-8062X, Santa Cruz Biotechnology, Santa Cruz, Calif.) were pre-incubated with cell extracts at 25° C. for 30 minutes prior to the HSE-binding reaction. After this preincubation, the binding reaction and analysis were performed as described above.

To determine whether the effects produced by the VSL#3 bacteria require viable bacteria and direct physical contact (e.g., as is necessary for type III secretion mechanisms) or are secreted products, VSL#3 was added to YAMC cells, as filter-sterilized conditioned medium or sonicated bacterial pellets, in a concentration-dependent manner (FIG. 19 panel B). Although live gram-negative bacteria and lipopolysaccharide (LPS), a cell wall component found only in gram-negative bacteria, can induce Hsp expression in epithelial cells (Kojima, 2003), the bacteria which comprise VSL#3 are all gram-positive organisms and thus none of them contain LPS. It was therefore of interest to discern whether any cell wall components of these gram-positive organisms possess Hsp-inducing potential. The Hsp induction produced by VSL#3 could be elicited in a dose-response fashion with the conditioned medium (“CM”) alone, indicating that neither direct contact nor live bacteria are necessary to elicit this response. Sonicated organisms (“pellet”) are unable to induce the heat shock response, indicating that the inducing factors are secreted products and not cell wall components.

Examination of two other cell lines revealed that the ability of probiotic-CM to induce Hsp72 expression is specific to epithelial cells (FIG. 19 panel C). MSIE is a murine small intestine epithelial cell line; these cells respond in a similar fashion as YAMC cells. In contrast, although 3T3 fibroblasts are able to mount a heat shock response to thermal stress, they do not respond to treatment with VSL#3-CM, indicating that the effects of VSL#3-CM are specific to epithelial cell types. Attempts to induce heat shock protein expression in all of these cell lines using E. coli DH5α-CM were unsuccessful.

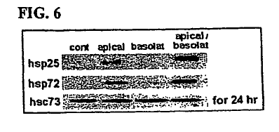

In addition, it was determined that the probiotic compounds induce intestinal epithelial heat shock proteins through apical (luminal) membrane-specific processes (see FIG. 6). When YAMC intestinal epithelial cells are grown on a permeable support, they form tight junctions and exhibit a high degree of polarity. Cells exposed to conditioned medium from the luminal side demonstrate robust Hsp25 and Hsp72 protein expression. Basolateral addition fails to stimulate a response. When added to both sides, VSL#3-CM has a similar effect to what is seen when it is added only to the apical side. These data suggest the presence of specific receptors or entry pathways for the probiotic-derived bioactive factors or agents.

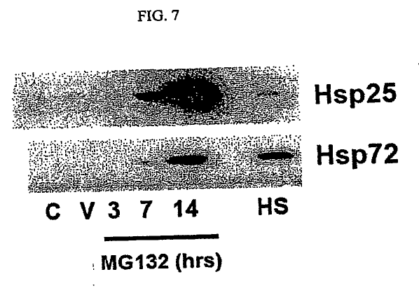

The proteasome inhibitor MG132 was then used to determine whether the time course of Hsp induction following proteasomal inhibition in epithelial cells would parallel the induction attributable to VSL#3-CM treatment. Treatment with MG132 results in a very strong induction of both Hsp25 and Hsp72 that is more robust than thermal stress (FIG. 7). A comparison of FIG. 7 with the time course of FIG. 19A shows that the appearance of a signal by 7 to 14 hours more closely parallels what is seen with VSL#3-CM than the time course normally observed with thermal stress, which induces Hsp expression by two hours in this cell line (Kojima, 2003). VSL#3-CM acting through a mechanism of proteasome inhibition would be expected to display a time course comparable to a known proteasome inhibitor such as MG132 and not as comparable to a mere physical stress such as heat shock.

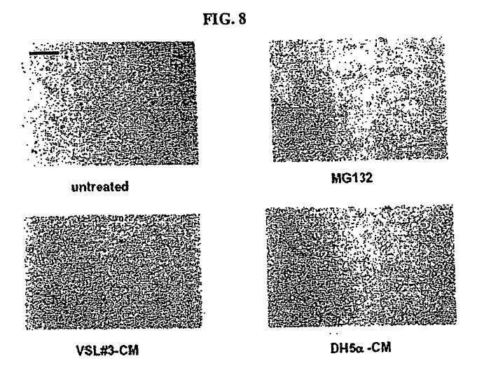

Unlike VSL#3-CM, which did not result in any change in viability compared to untreated control cells even after 24 hours of treatment, prolonged exposure of YAMC cells to MG132 resulted in markedly increased cell death, suggesting that MG132 is significantly more toxic than VSL#3-CM (FIG. 8).

### a. Time Course of Heat Shock Protein Expression Induction

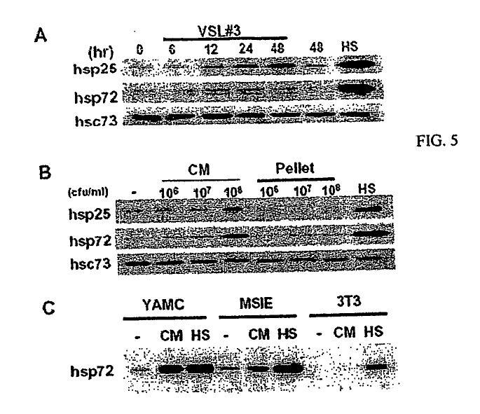

Probiotics induce heat shock proteins in intestinal epithelial cells. It has been shown that enteric flora (luminal bacteria of the colon) in the gut influence expression of epithelial heat shock proteins. Proteasome inhibitors are potent inducers of heat shock protein expression through activation of the heat shock transcription factor, HSF-1. Based on these observations, corroboration of the above findings was sought by determining whether induction of heat shock protein expression occurred. YAMC cells were co-cultured with the probiotic VSL#3 to test its ability to induce heat shock protein expression. By immunoblot analysis, VSL#3 was shown to induce Hsp25 and Hsp72 expression in YAMC cells beginning at 6 to 12 hours (FIG. 5 panel A). Expression of the constitutively expressed heat shock protein Hsc73, serving as a loading control, was not affected by VSL#3. Untreated cells left in culture for 48 hours do not mount an Hsp response, demonstrating that the effect is specific to the probiotic treatment and not a time-dependent characteristic of the cells grown in culture.

To determine whether the effects produced by the VSL#3 bacteria require viable bacteria and direct physical contact (e.g., as is necessary for type III secretion mechanisms) or are secreted bacterial products, VSL#3 was added to YAMC cells, as filter-sterilized conditioned medium or sonicated bacterial pellets, in a concentration-dependent manner (FIG. 5 panel B). Although live gram-negative bacteria and lipopolysaccharide (LPS), a cell wall component found only in gram-negative bacteria, can induce Hsp expression in epithelial cells, the bacteria which comprise VSL#3 are all gram-positive organisms and, thus, lack LPS. It was therefore of interest to discern whether any cell wall components of these gram-positive organisms possess Hsp-inducing potential. The Hsp induction produced by VSL#3 could be elicited in a dose-response fashion with the conditioned medium (“CM”) alone, indicating that neither direct contact nor the presence of live bacteria are necessary to elicit this response, Sonicated organisms (“pellet”) are unable to induce the heat shock response, leading to the expectation that the inducing factors are secreted products and not cell wall components. Examination of two other cell lines revealed that the ability of probiotic-CM to induce Hsp72 expression is specific to epithelial cells (FIG. 5 panel C). MSIE is a murine small intestine epithelial cell line; these cells respond in a fashion similar to YAMC cells. In contrast, although 3T3 fibroblasts are able mount a heat shock response to thermal stress, they do not respond to treatment with VSL#3-CM. Thus, it is expected that the effects of VSL#3-CM are specific to epithelial cell types. Attempts to induce heat shock protein expression in all of these cell lines using E. coli DH5α-CM were unsuccessful.

The data establish that a compound derivable from VSL#3-CM exhibits a cytoprotective function by inducing the expression of heat shock proteins, in addition to exhibiting an anti-inflammatory function. Accordingly, the invention contemplates an isolated, anti-inflammatory, cytoprotective compound derivable from VSL#3-CM, related compositions such as pharmaceutical compositions and kits comprising the compound(s), as well as methods of producing the compound(s) and methods of using the compound(s) to prevent or treat an inflammatory disorder such as IBD or to ameliorate a symptom of such a disorder.

### b. Hsp Expression Induction is Mediated by HSF-1 Activation

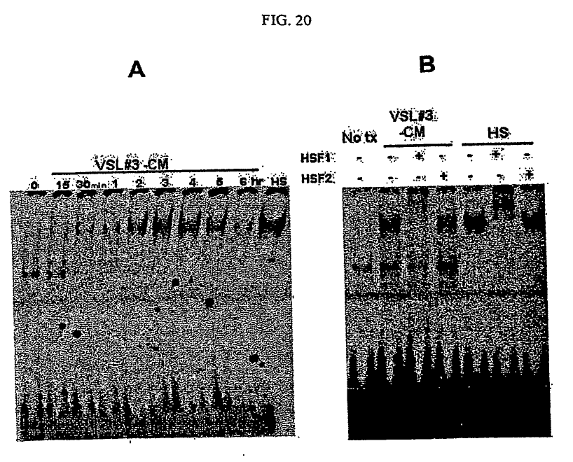

Electrophoretic mobility shift assays were performed to determine whether or not the induction of Hsp expression by VSL#3-CM was transcriptional in nature. From FIG. 20 it can be seen that VSL#3-CM induces binding of the heat shock transcription factor HSF, reaching a maximal binding signal around 4 or 5 hours after exposure and then tapering off after 6 hours (panel A). Specificity of this binding was confirmed using antibodies against the transcription factors HSF-1 and HSF-2 (panel B), which demonstrates that the major transcription factor involved in Hsp induction by VSL#3-CM is HSF-1; HSF-2 does not appear to play a role. The time course of Hsp induction by the proteasome inhibitor MG132 is similar to that produced by probiotics, but MG132 is more toxic. As the proteasome time course data indicated that VSL#3-CM acted quickly like other proteasome inhibitors such as MG132 (see FIG. 18), we determined, using MG132, whether the time course of Hsp induction following proteasomal inhibition in epithelial cells would parallel the same kinetics as observed with VSL#3-CM treatment. Treatment with MG132 resulted in a very strong induction of both Hsp25 and Hsp72 and the induction was more robust than that resulting from thermal stress (FIG. 7). A comparison of FIG. 7 with the time course of FIG. 19A shows that the appearance of a signal by 7 to 14 hours more closely parallels what is seen with VSL#3 than the time course normally observed with thermal stress, which induces Hsp expression by two hours in this cell line. If VSL#3-CM were acting through a mechanism of proteasome inhibition, it would display a time course more comparable to a known proteasome inhibitor such as MG132 and less similar to a mere physical stress such as heat shock. Unlike VSL#3, which did not result in any change in viability compared to untreated control cells even after 24 hours of treatment, prolonged exposure of YAMC cells to MG132 resulted in markedly increased cell death, suggesting that MG132 is significantly more toxic than VSL#3-CM.

### EXAMPLE 9

### Probiotics Protect Intestinal Epithelial Cells Against Oxidant Stress

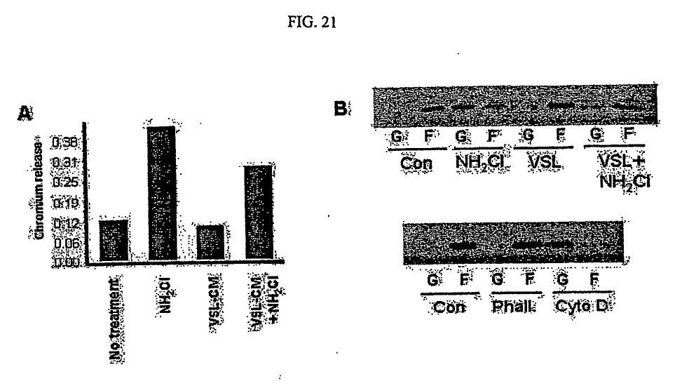

To determine whether VSL#3-CM protects gut epithelial cells from injury, the oxidant monochloramine (NH₂Cl) was used. Monochloramine is a pathophysiologically relevant oxidant produced in large quantities when hypochlorous acid, released from innate immune cells within inflamed tissues, reacts with ammonia in vivo. Once formed, monochloramine causes loss of tight junction barrier function, mitochondrial injury, cytoskeletal disruption, impaired membrane transport functions, and eventual cell death. Cells were treated with VSL#3-CM overnight and, after exposure to monochloramine, cell viability was assessed using ⁵¹Cr release (FIG. 21, panel A).

YAMC cells were grown in 24-well plates and either left untreated (control), or treated with VSL#3-CM overnight. Cells were loaded with ⁵¹Cr (50 μCi/ml; Sigma Chemical Co.; 250 μl/well in a 24-well plate format, or 12.5 μCi per well) for 60 minutes, washed, and incubated in medium with 0.6 mM of the oxidant monochloramine to induce cell injury. Medium was harvested after 60 minutes and the ⁵¹Cr remaining in the cells extracted with 1N HNO₃ for 4 hours. ⁵¹Cr in the released and cellular fractions was counted by liquid scintillation spectroscopy. ⁵¹Cr released was calculated as amount released divided by released plus cellular remainder.

At 0.6 mM NH₂Cl, VSL#3-CM pretreatment results in a mild but statistically significant protective effect, decreasing the NH₂Cl-stimulated ⁵¹Cr release and improving epithelial cell viability in the face of oxidant injury by about one-third compared to control cells treated with monochloramine alone (P<0.05).

As another functional readout, the ability of VSL#3-CM treatment to protect epithelial cells against cytoskeletal damage from oxidant stress was determined using F/G actin assays. Filamentous actin (F-actin) carries out important functions involved in the maintenance of cellular scaffolding and shape, as well as acting as an anchoring point for numerous integral membrane proteins. Nevertheless, the actin cytoskeleton is particularly vulnerable to injury which can result in cellular compromise. Exposure to monochloramine causes rapid dissociation of filamentous actin. Hence, we determined whether VSL#3-CM treatment would protect the integrity of cytoskeletal filamentous actin in the face of oxidant stress.

F/G actin assays were conducted by initially shifting confluent YAMC cell monolayers to 37° C. in IFN-γ-free medium and treating with VSL#3-CM overnight. Prior to assessment, cells were treated with phalloidin (30 μg/ml for 2 hours; Molecular probes, Eugene, Oreg.), cytochalasin D (10 μg/ml for 15 min), or the oxidant monochloramine (0.6 mM for 30 minutes). Cells were rinsed in PBS, harvested, centrifuged (14,000×g for 20 seconds at room temperature) and the pellets resuspended in 200 μl of 30° C. lysis buffer (1 mM ATP, 50 mM PIPES, pH 6.9, 50 mM NaCl, 5 mM MgCl₂, 5 mM EGTA, 5% (vol/vol) glycerol, 0.1% (vol/vol) Nonidet P-40, Tween 20, and Triton X-100, containing complete protease inhibitor cocktail). Cells were homogenized by gently pipetting up and down ten times and incubated at 30° C. for 10 minutes. Samples were centrifuged at 100,000×g for 60 minutes at 30° C. and the supernatants were removed for determination of G actin content. Pellets containing F-actin were resuspended in 200 μl of 4° C. distilled water with 1 μM cytochalasin D and left on ice for 60 minutes. Then, 20 μl of each extraction was removed, 6 μl 3× Laemmli stop solution was added and the samples were heated to 65° C. for 10 minutes. Samples were resolved by 12.5% SDS-PAGE and immediately transferred to PVDF membranes. After transfer, analysis of actin was performed using a polyclonal anti-actin antiserum by Western blotting (Cytoskeleton, Denver, Colo.). Since the F-actin fraction has been depolymerized, only the monomeric 45 kDa form is observed on the Western blots.

Monochloramine treatment alone (0.6 mM) causes a disruption of F-actin filaments, as demonstrated by a decrease in F-actin and an increase in G-actin (FIG. 21, panel B). By itself, VSL#3-CM has little effect on the F/G actin distribution. However, YAMC cells pretreated with VSL#3-CM prior to NH₂Cl exposure demonstrated significantly less change in the F/G actin distribution, again indicating that this probiotic provides some protection against oxidant injury. As positive and negative controls, phalloidin, (which binds and stabilizes the barbed ends of F-actin filaments, thus increasing the amount of F-actin) and cytochalasin D (an F-actin disrupting agent which greatly increases the amount of globular (G) actin) were used (panel B).

The results of the experiment disclosed in this example are consistent with VSL#3-CM exhibiting reduced toxicity relative to known proteasome inhibitors such as MG132, and also are consistent with the disclosure herein that VSL#3-CM induces the expression of at least one heat shock protein in epithelial cells, in establishing a cytoprotective role for at least one compound derivable from VSL#3-CM.

### EXAMPLE 10

### Properties of Bioactive Probiotic Agents

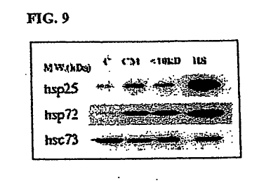

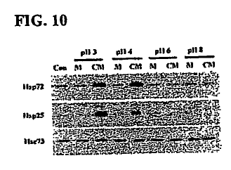

As shown in FIG. 9, the majority of bioactivity found in, or derived from, VSL#3-CM appears to reside in fractions that are less than 10 kDa. Fractions were prepared through Centricon filters with specific molecular weight cutoffs.

Another property of the bioactivity of the VSL#3-CM is its pH-sensitivity (FIG. 0). The pH of conditioned medium prior to addition to the apical side of YAMC cells (which results in a 1:10 dilution) is critical. This ultimately affects the final pH of the bathing medium, lowering it to between 6.5 and 7.0. The data, therefore, indicate that any active probiotic protein factors are more active in the acid microclimate that exists in the unstirred water layer above the luminal membrane of intestinal epithelial cells where pH ranges between 6.5-7.0.

Additionally, the bioactive agent(s) in VSL#3-CM were subjected to protease assay by exposure of VSL#3-CM to pepsin using a standard protocol known in the art. The results revealed that pepsin did not affect the bioactivity of VSL#3-CM, indicating that the bioactive agent(s) were not peptides or proteins susceptible to pepsin digestion. It is expected that the bioactive agent(s) are non-proteinaceous compounds, such as non-polar compounds like organic acids; of course, the bioactive agent(s) could be peptides, such as relatively small peptides, that are refractory to pepsin digestion or that retain an active peptide fragment following exposure to pepsin.

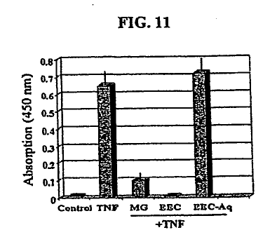

The proteasome inhibitors in VSL#3 may be small organic molecules. Some proteasome inhibitors found in nature are small molecular weight organic esters or organic acid derivatives, such as those in green tea. An ether extraction of the VSL#3-CM was undertaken to determine if bioactive factors could be concentrated to produce a more consistent and robust response. As shown in FIG. 11, the effects of ether-extracted compounds (EEC) and MG132 on NF-κB activity were determined using an NF-κB ELISA assay (Active Motif). TNF-α stimulation (30 ng/ml) alone caused a significant increase in NF-κB activation (second bar from left). Both MG132 and EEC significantly inhibited TNF-α-stimulated NF-κB activity (third and fourth bars from left). In contrast, the remaining aqueous phase following separation from the ether fraction was devoid of activity (far right bar).

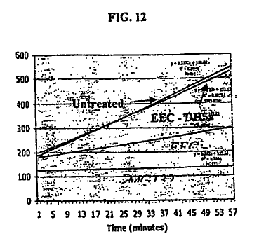

To determine if EEC directly inhibit proteasomal function, the in vitro activity of the 20S proteasomal component (barrel) provided by the commercial proteasomal assay (Calbiochem) was examined in the presence and absence of EEC from VSL#3 and E. coli (DH5α). As shown in FIG. 12, proteasomal function was unaffected by EEC from DH5α (compare slopes). In contrast, there was significant inhibition of in vitro proteasomal activity by EEC from VSL#3 and by MG132. These data support the expectation that VSL#3 EEC enter the cell intact through a specific apical membrane process, subsequently acting directly on cellular proteasomal function

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein with the same or similar results being achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Annuk et al., J. Appl. Microbiol., 94:403-412, 2003.
Arvola et al., Pediatrics, 104:e64, 1999.
Beck, Shock, 3:398-402, 1995.
Besnard et al., Gut, 43(5):634-638, 1998.
Boone et al., Inflamm. Bowel Dis., 8:201-212, 2002.
Braude and Siemienski, J. Clin. Invest., 47:1763-1773, 1968.
Campierei et al., Gastroenterology, 118 A4179, 2000.
Chang, In: Inflammatory Bowel Diseases, Kirsner (Ed.), Philadelphia, W. B. Saunders Company, 3-19, 1999.
Cleveland et al., Int. J. Food Microbiol., 71:1-20, 2001.
Gao, J. Clin. Invest., 106:439-448, 2000.
Gionchetti et al., Gastroenterology, 119:305-309, 2000b.
Gionchetti et al., Gastroenterology, 124:1202-1209, 2003.
Gionchetti et al., J. Gastroenterol. Hepatol., 15:489-493, 2000a.
Isolauri et al., Pediatrics, 88:90-97. 1001.
Jobin and Sartor, Inflamm. Bowel Dis., 6:206-213, 2000.
Kisselev and Goldberg, Chem. & Biol., 8:739-758, 2001.
Kojima, Gastroenterology, 124:1395-1407, 2003.
Kullisaar et al., Int. J. Food Microbiol., 72:215-224, 2002.
Lee and Goldberg, Mol. Cell. Biol., 18:30-38, 1998.
Lee et al., Appl. Environ. Microbiol., 66:3692-3697, 2000.
Lee et al., J. Med. Microbiol., 52:925-930, 2003.
Leiper et al., Baillieres Clin. Gastroenterol., 12(1):179-99, 1998.
Liu et al., Am. J. Physiol., 284:C1073-1082, 2003.
Maassen et al., Vaccine, 18:2613-2623, 2000.
Madsen et al., Gastroenterology, 121:580-591, 2001.
Majamaa et al., J. Pediatr. Gastroenterol. Nutr., 20:333-338, 1995.
Makowiec et al., Z. Gastroenterol., 36(8):619-24, 1998.
Musch et al., Am. J. Physiol., 270:C429-436, 1996.
Musch et al., Gastroenterology, 117:115-122, 1999.
Myung et al., Med. Res. Rev., 21(4):245-273 (2001).
Neish, Science, 289:1560-1563, 2000.
Neurath et al., Ann. NY Acad. Sci., 859:149-159, 1998.
Neurath et al., Gut, 43:856-860, 1998.
Neurath et al., Nat. Med., 2:998-1004, 1996.
Niedzielin et al., Eur. J. Gastroenterol. Hepatol., 13:1143-1147, 2001.
Ocana et al., Curr. Microbiol., 38:279-284, 1999.
Paraje et al., New Microbiol., 23:423-431, 2000.
Parsell and Lindquist, Annu. Rev. Genet., 27:437-496, 1993.
Pirkkala, Mol. Cell Biol., 20:2670-2675, 2000.
Podolsky, N. Engl. J. Med., 347:417-429, 2002.
Remington's Pharmaceutical Sciences, 15^{th} ed., pages 1035-1038 and 1570-1580, Mack Publishing Company, Easton, Pa., 1980.
Ropeleski et al., Gastroenterology, 124:1358-1368, 2003.
Schmid and Adler, Gastroenterology, 118:1208-1228, 2000.
Schmitz et al., Gastroenterology, 116:301-309, 1999.
Smith, Anal. Biochem., 150:76-85, 1985.
Urayama et al., J. Clin. Invest., 102:1860-1865, 1998.
Voges, Annu. Rev. Biochem., 68:1015-1068, 1999.
Whitehead et al., Proc. Natl. Acad. Sci. USA, 90:587-591, 1993.
Wollowski et al., Amer. J. Clinical Nutrition, 73:451S-455S, 2001.

What is claimed is:

1. A composition comprising an isolated anti-inflammatory, cytoprotective compound.

2. The composition of claim 1, wherein the compound is present in an ether-extracted fraction of the probiotic-conditioned medium.

3. The composition of claim 2, wherein the compound is an organic acid.

4. The composition of claim 1, wherein the compound induces the expression of at least one heat shock protein.

5. The composition of claim 4, wherein the heat shock protein is selected from the group consisting of Hsp25 and Hsp72.

6. The composition of claim 1, wherein the compound is an inhibitor of NF-κB activation.

7. The composition of claim 6, wherein the compound inhibits NF-κB activation by stabilizing IκB.

8. The composition of claim 1, wherein the compound is a proteasome inhibitor.

9. The composition of claim 8, wherein the proteasome inhibitor selectively inhibits the chymotrypsin-like activity of the proteasome.

10. The composition of claim 8, wherein the proteasome inhibitor selectively inhibits the proteasome in an epithelial cell.

11. The composition of claim 10, wherein the epithelial cell is an intestinal epithelial cell.

12. The composition of claim 1, wherein the probiotic-conditioned medium is VSL#3-conditioned medium.

13. A method for treating a patient with an inflammatory disorder comprising administering to the patient an effective amount of an isolated anti-inflammatory, cytoprotective compound derived from a probiotic-conditioned medium.

14. The method of claim 13, wherein the probiotic-conditioned medium is VSL#3-conditioned medium.

15. The method of claim 13, wherein the inflammatory disorder is an inflammatory bowel disease.

16. The method of claim 15, wherein the inflammatory bowel disease is Crohn's disease.

17. The method of claim 15, wherein the inflammatory bowel disease is ulcerative colitis.

18. The method of claim 13, wherein the compound is derived from an ether-extracted fraction of the medium.

19. The method of claim 18, wherein the compound is an organic acid.

20. The method of claim 13, wherein the compound induces the expression of at least one heat shock protein.

21. The method of claim 20, wherein the heat shock protein is selected from the group consisting of Hsp25 and Hsp72.

22. The method of claim 13, wherein the compound is an inhibitor of NF-κB activation.

23. The method of claim 22, wherein NF-κB activation is inhibited by stabilizing IκB.

24. The method of claim 13, wherein the compound is an inhibitor of a protease activity.

25. The method of claim 24, wherein the inhibitor selectively inhibits a protease activity of a proteasome in an epithelial cell.

26. The method of claim 25, wherein the inhibitor selectively inhibits the chymotrypsin-like activity of the proteasome.

27. The method of claim 26, wherein the epithelial cell is an intestinal epithelial cell.

28. A pharmaceutical composition comprising an isolated anti-inflammatory, cytoprotective compound derived from a probiotic-conditioned medium and at least one pharmaceutically acceptable excipient.

29. The pharmaceutical composition of claim 28, wherein the compound is derived from an ether-extracted fraction of the medium.

30. The pharmaceutical composition of claim 29, wherein the compound is an organic acid.

31. The pharmaceutical composition of claim 28, wherein the compound induces the expression of at least one heat shock protein.

32. The pharmaceutical composition of claim 31, wherein the heat shock protein is selected from the group consisting of Hsp25 and Hsp72.

33. The pharmaceutical composition of claim 28, wherein the compound is an inhibitor of NF-κB activation.

34. The pharmaceutical composition of claim 33, wherein the compound inhibits NF-κB activation by stabilizing IκB.

35. The pharmaceutical composition of claim 28, wherein the compound is a proteasome inhibitor.

36. The pharmaceutical composition of claim 35, wherein the proteasome inhibitor selectively inhibits a protease activity of a proteasome in an epithelial cell.

37. The pharmaceutical composition of claim 35, wherein the proteasome inhibitor selectively inhibits the chymotrypsin-like activity of the proteasome.

38. The pharmaceutical composition of claim 37, wherein the epithelial cell is an intestinal epithelial cell.

39. The pharmaceutical composition of claim 28, wherein the probiotic-conditioned medium is VSL#3-conditioned medium.

40. A method of producing an isolated, anti-inflammatory, cytoprotective compound comprising,
obtaining a VSL#3-conditioned medium; and
isolating an anti-inflammatory, cytoprotective compound from the VSL#3-conditioned medium, thereby producing an isolated, anti-inflammatory, cytoprotective compound.

41. A method of screening for a modulator of monocyte chemoattractant protein-1 (MCP-1) release, comprising:
(a) combining a candidate modulator, a probiotic-conditioned medium, and an epithelial cell;
(b) measuring MCP-1 release by said cell; and
(c) comparing the MCP-1 release in the presence, and absence, of said candidate modulator, wherein a difference in said MCP-1 release identifies the candidate modulator as a modulator of MCP-1 release.

42. The composition of claim 7, wherein the stabilized IκB is phosphorylated IκBα.

43. The method of claim 13, wherein the anti-inflammatory, cytoprotective compound does not alter the ubiquitination level of at least one protein amenable to ubiquitination in an epithelial cell exposed to said compound.

44. A method of preventing an inflammatory disorder comprising administering an effective amount of an isolated, anti-inflammatory, cytoprotective compound derived from a probiotic-conditioned medium.

45. A method of screening for a modulator of heat shock protein expression, comprising
(a) combining a candidate modulator, a probiotic-conditioned medium, and an epithelial cell;
(b) measuring heat shock protein expression in said cell; and
(c) comparing the heat shock protein expression in the presence, and absence, of said candidate modulator, wherein a difference in said heat shock protein expression identifies the candidate modulator as a modulator of heat shock protein expression.

46. The method of claim 45 wherein said heat shock protein is selected from the group consisting of Hsp25 and Hsp72.

47. The method of claim 45 wherein said modulator alters the activity of Heat Shock Transcription Factor-1 (HSF-1).

48. A kit for treating or preventing an inflammatory disorder comprising a pharmaceutical composition according to claim 28 and instructions for administration of said composition to treat or prevent said disorder.
